(19) United States
(12) Patent Application Publication
(10) Pub. No.: US 2025/0161302 A1
(43) Pub. Date: May 22, 2025

(54) EXTENDED RELEASE UPADACITINIB FORMULATIONS

(71) Applicant: ABBVIE INC., North Chicago, IL (US)

(72) Inventors: Yihong QIU, Vernon Hills, IL (US); Ye HUANG, Gurnee, IL (US); Deliang ZHOU, Vernon Hills, IL (US)

(21) Appl. No.: 19/027,247

(22) Filed: Jan. 17, 2025

Related U.S. Application Data
(63) Continuation of application No. 18/259,744, filed on Jun. 28, 2023.
(60) Provisional application No. 63/131,564, filed on Dec. 29, 2020.

(51) Int. Cl. A61K 31/4985; A61K 9/00; A61K 9/20; A61K 9/28

(52) U.S. Cl. CPC A61K 31/4985; A61K 9/0004; A61K 9/2009; A61K 9/2013; A61K 9/2018; A61K 9/2031; A61K 9/205; A61K 9/2054; A61K 9/2072; A61K 9/2086; A61K 9/2853; A61K 9/2866

(57) ABSTRACT

The disclosure describes extended release solid dosage forms comprising upadacitinib, or a pharmaceutically acceptable salt thereof, wherein the solid dosage form provides pH-independent drug release. In particular, the disclosure describes extended release solid dosage forms comprising upadacitinib, or a pharmaceutically acceptable salt thereof, at least one pH-dependent polymer and at least one release control material.

### CROSS REFERENCE TO RELATED APPLICATIONS

This application claims priority to U.S. Patent Application No. 63/131,564, filed on Dec. 29, 2020, the entire contents of which are fully incorporated herein by reference.

### BACKGROUND

Upadacitinib is a Janus kinase (JAK) inhibitor marketed in the United States for the treatment of adults with moderately to severely active rheumatoid arthritis (RA) who have had an inadequate response or intolerance to methotrexate under the tradename RINVOQ. The marketed product is a once daily extended release tablet comprising tartaric acid as an acidic pH modifier and hydroxypropylmethylcellulose (HPMC) as a release control polymer. In addition to the 15 mg upadacitinib dose being marketed for the treatment of RA, approval for the 15 mg dose is also sought for the treatment of active psoriatic arthritis and active ankylosying spondylitis. A lower dose (7.5 mg) will be marketed in Japan. Higher doses (30 mg and 45 mg) are respectively planned for the treatment of atopic dermatitis and IBD diseases, such as Crohn's disease and ulcerative colitis. The tablet size for each of these solid dosage forms is nearly 500 mg, which may impede swallowability, particularly in patients with difficulty swallowing, such as pediatric, juvenile and/or elderly patients. Furthermore, if the tablet is not stored properly (e.g., under low humidity), the appearance, dissolution rate, and impurity profile have been found to be negatively impacted. Thus, there exists a need for improved solid dosage forms comprising upadacitinib that retain desirable characteristics of the marketed product, such as a similar dissolution profile, but without the negative characteristics, such as the swallowability issues and/or storage problems.

### SUMMARY

Provided herein are improved extended release solid dosage forms comprising upadacitinib, or a pharmaceutically acceptable salt thereof.

In one aspect, the present disclosure provides an extended release solid dosage form comprising upadacitinib, or a pharmaceutically acceptable salt thereof, wherein the solid dosage form provides pH-independent drug release.

In one embodiment, the solid dosage form comprises a matrix system. In some such embodiments, the matrix system comprises a pH-dependent polymer. In some such embodiments, the solid dosage form further comprises at least one release control material. In some such embodiments, the solid dosage form comprises less than 10% by weight of a hygroscopic acidic pH modifier.

In one embodiment, the solid dosage form comprises a release rate modifier, preferably the release rate modifier is not a hygroscopic acidic pH modifier. In some such embodiments, the release rate modifier is an ion exchange resin. In some such embodiments, the release rate modifier is a non-acidic or a basic pH modifier, such as sodium carbonate, meglumine, tribasic sodium phosphate dodecahydrate (Na₃PO₄·12 H₂O), sodium hydroxide, sodium bicarbonate, magnesium oxide, potassium hydroxide, or calcium phosphate. In some such embodiments, the solid dosage form further comprises an anionic polymer or an anionic polysaccharide, such as hydroxypropyl methyl cellulose phthalate (HPMCP), cellulose acetate phthalate (CAP), hydroxypropyl methylcellulose acetate succinate (HPMCAS), polyvinylacetate phthalate (PVAP), methacrylic acid copolymers (Eudragit L), alginic acid, pectin, hyaluronic acid, or carboxymethylcellulose.

In one embodiment, the solid dosage form comprises a barrier layer covering a portion of the release surface of the solid dosage form. In some such embodiments, the barrier layer comprises a polymer, preferably a pH-dependent polymer, that acts as a coating to cover a portion of the release surface of the solid dosage form. In some such embodiments, a pH-dependent barrier layer is applied to a partial surface of the dosage form (e.g., a tablet) using solvent based coating or compression coating processes. In some such embodiments, the solid dosage form further comprises a release rate modifier. In some such embodiments, the release rate modifier is an acidic pH modifier such as fumaric acid.

In one embodiment, the solid dosage form is an osmotic pump drug release system. In some such embodiments, the osmotic pump drug release system comprises a release rate modifier. In some such embodiments, the release rate modifier is an acidic pH modifier such as fumaric acid. In other such embodiments, the osmotic pump drug release system does not comprise a release rate modifier.

### BRIEF DESCRIPTION OF THE DRAWINGS

FIGS. 1A-1B depict the effects of tartaric acid present in uncoated (FIG. 1A) and coated (FIG. 1B) upadacitinib-containing tablets. In FIG. 1A, increasing mottling/deliquesence is observed in uncoated tablets containing increasing amounts of tartaric acid (TA), i.e., at 0%, 10%, 20%, and 30% TA with a moisture content of 4.2%, when stored at 30° C./53% relative humidity (RH) for 2 months. In FIG. 1B, a visual comparison of “unstressed” 7.5 mg RINVOQ coated tablets of Table 1 versus “stressed” 7.5 mg RINVOQ coated tablets (subjected 40° C./53% RH for >2 months) shows differences in dissolution in a pH 6.8 50 mM sodium phosphate buffer medium. Without being bound by any particular theory, it is believed that the mottling and differences in dissolution is caused by two main respective factors: (a) solubilization of the hydroscopic organic acid (tartaric acid) in water leading to deliquescence and leaching of tartaric acid into the film coat, such as shown in FIG. 1A, and (b) cross-linking of the solubilized tartaric acid with the film coat containing polyvinylalcohol (PVA), leading to changes in the dissolution, such as shown by the changes in dissolution of stressed versus unstressed tablets in FIG. 1B.

FIG. 2 depicts a reaction scheme for the formation of the non-genotoxic upadacitinib hydroxymethyl impurity (UHM) impurity from the reaction of upadacitinib in the presence of acid, water, and formaldehyde.

FIG. 3 compares the dissolution profile of RINVOQ (30 mg) 500 mg tablet at pH 1.1 and pH 6.8 to that of smaller sized (200 mg) tablets with tartaric acid (T1) and without tartaric acid (A1).

FIGS. 4-8 compare the dissolution profile of RINVOQ (30 mg) 500 mg tablet at pH 1.1 and pH 6.8, containing tartaric acid and a release control material (HPMC), to that of the smaller sized tablets, Formulation AS1 (FIG. 4), Formulation AS2 (FIG. 5), Formulation AS3 (FIG. 6), Formulation AS4 (FIG. 7), and Formulation AS5 (FIG. 8), each containing an enteric polymer (HPMCAS) and a release control material (HPMC).

FIGS. 9-11 compare the dissolution profile of RINVOQ (30 mg) 500 mg tablet at pH 1.1 and pH 6.8, containing tartaric acid and a release control material (HPMC), to that of the smaller sized tablets, Formulation AL1 (FIG. 9), Formulation AL2 (FIG. 10), Formulation AL3 (FIG. 11), each containing the anionic polysaccharide alginic acid and a release control material (HPMC).

FIG. 12 compares the dissolution profile of RINVOQ (30 mg) 500 mg tablet at pH 1.1 and 6.8, containing tartaric acid and a release control material (HPMC), to that of a smaller sized tablet, Formulation T2, which contains an enteric polymer (HPMCAS), a release control material (HPMC), and a hygroscopic acidic pH modifier (20% tartaric acid).

FIG. 13 compares the dissolution profile of a 30 mg RINVOQ tablet to that of a 15 mg Formulation E1 tablet in (1) 0.1N HCl and (2) pH 6.8 buffer.

FIG. 14 compares the dissolution profile of 30 mg Formulation E2, E3, and E4 tablets in (1) 0.1N HCl and (2) pH 6.8 buffer.

FIG. 15 compares the dissolution profile of 30 mg Formulation E4 and E5 tablets in (1) 0.1N HCl and (2) pH 6.8 buffer.

FIG. 16 compares the dissolution profile of 30 mg Formulation E6 and E7 tablets in (1) 0.1N HCl and (2) pH 6.8 buffer.

FIG. 17 compares the dissolution profile of 30 mg Formulation E6 and E8 tablets in (1) 0.1N HCl and (2) pH 6.8 buffer.

FIG. 18 compares the dissolution profile of 11.52 mg Formulation E9, E10, and E11 tablets in (1) 0.1N HCl and (2) pH 6.8 buffer.

FIG. 19 compares the dissolution profile of 11.52 mg Formulation E10 and E12 tablets in (1) 0.1N HCl and (2) pH 6.8 buffer.

### DETAILED DESCRIPTION OF CERTAIN EMBODIMENTS

Extended release solid dosage forms comprising upadacitinib ((3S,4R)-3-ethyl-4-(3H-imidazo[1,2-a]pyrrolo[2,3-e]pyrazin-8-yl)-N-(2,2,2-trifluoroethyl) pyrrolidine-1-carboxamide) or a pharmaceutically acceptable salt thereof, an acidic pH modifier, such as tartaric acid, and a release control polymer, such as hydroxypropyl methylcellulose (HPMC), are described in WO 2017/066775, and encompass the solid dosage form that is marketed as RINVOQ (upadacitinib). As noted and understood from the '775 publication, as the solid dosage form begins to erode upon administration and exposure to water in the stomach, the acidic pH modifier and upadacitinib solubilize and form, together with the release control polymer, an acidic gel microenvironment, allowing for the extended release of upadacitinib from the gel in the solid dosage form at a relatively constant rate despite external macroenvironmental pH changes. Such an extended release profile is noted to be particularly advantageous, since the pH of the gastrointestinal tract may vary significantly from the stomach (e.g., pH of about 1.5-3), to the duodenum (e.g., pH of about 4-5), to the lower part of the small intestines (e.g., pH of about 6.5-7.5).

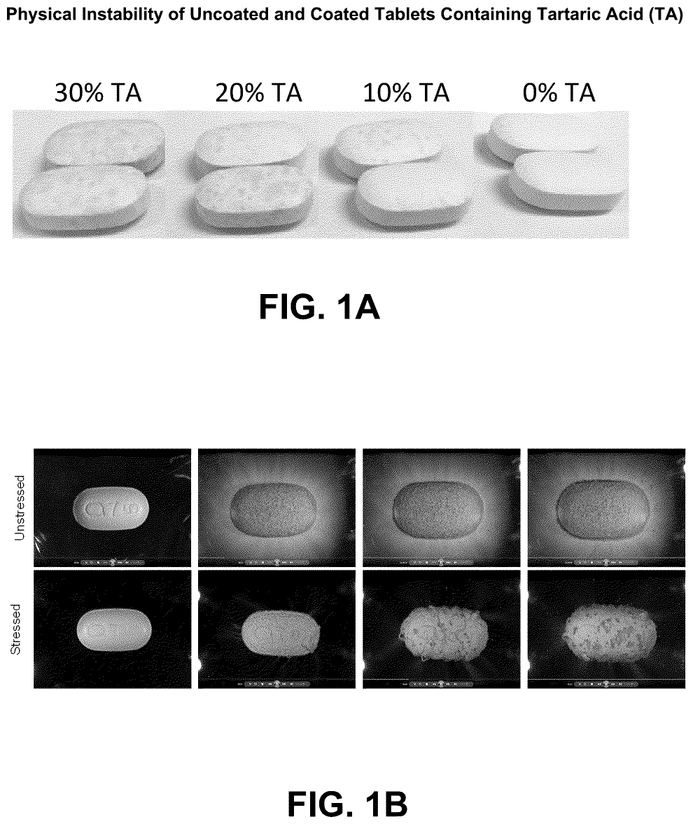

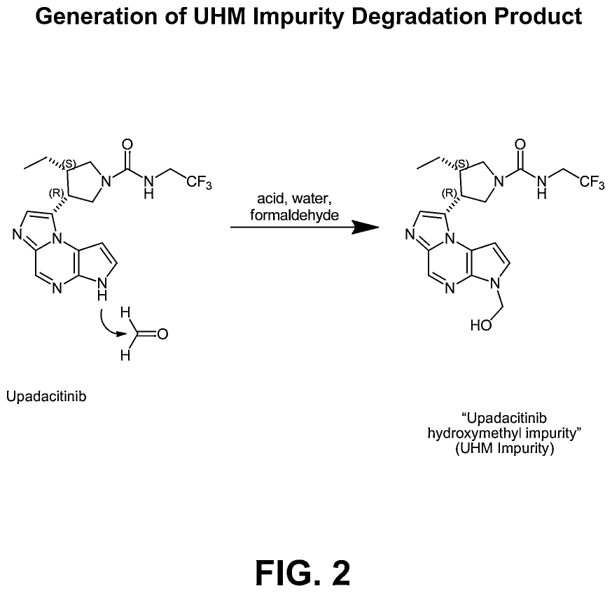

Several disadvantages have now been identified with the currently marketed solid dosage form, particularly related to storage and handling. For example, as storage time increases without appropriate moisture protection, the tablet appearance grows increasingly mottled (see FIG. 1), the dissolution rate of upadacitinib decreases, and levels of a non-genotoxic upadacitinib hydroxymethyl impurity (UHM Impurity) (see FIG. 2) increase. Without being bound by any particular theory, these challenges in the storage and handling of the marketed RINVOQ tablets may be caused in part by the presence of a relatively high amount (e.g., 20%) of the hygroscopic organic acid, tartaric acid. For example, over time, and especially under conditions which do not maintain a low water environment, water is absorbed into the tablet (facilitated by the water loving nature of tartaric acid), leading to increasingly solubilized tartaric acid. The tablet mottling may be due to either deliquescence and leaching of the solubilized tartaric acid through the film coat and/or cross-linking reaction of the solubilized tartaric acid with the film coat containing polyvinyl alcohol (PVA). The decreased dissolution rate following storage, particularly under elevated temperature and humidity, may be attributed to increasingly solubilized tartaric acid reacting with PVA as a cross-linker. Increased amounts of solubilized tartaric acid may also be responsible for the increased amounts of the UHM impurity, which may be formed via a tartaric acid-catalyzed reaction of upadacitinib with trace formaldehyde that is present in some excipients, such as polyethylene glycol (PEG), HPMC, and PVA. Keeping the UHM Impurity at low levels to meet quality specifications for the marketed product (e.g., no more than about 0.5% w/w, or between about 0.1% to about 0.5% w/w, of the UHM impurity) requires maintaining a low water content of the tablet (e.g., no more than about 4% w/w, or between about 1% to about 4% w/w) for its shelf-life of 24 months. Drying the tablet followed by storage under very dry conditions (e.g., using a desiccant or sealed in aluminum foil packaging) is not a perfect solution.

Furthermore, in an effort to reduce the size and thereby improve the swallowability of the marketed product, it was discovered that use of tartaric acid as an acidic pH modifier in smaller tablets resulted in tablets with incomplete release compared to the marketed product RINVOQ.

The present disclosure provides new solid dosage forms comprising upadacitinib or a pharmaceutically acceptable salt thereof wherein the solid dosage forms provide pH-independent drug release.

Such solid dosage forms may allow for the reduction or even elimination of certain acidic pH modifiers, more specifically, hygroscopic organic acids, such as tartaric acid, in the formulation. Lower levels of hygroscopic acid in the formulation may improve the storage stability issues, leading to reduction of mottling, reduction of degradation products (such as the UHM Impurity), while retaining a similar release profile to the marketed upadacitinib product. Furthermore, lower levels of hygroscopic acids in the tablet allows for ease of manufacture, requiring less fillers and other excipients to compensate for its inclusion, thus providing a dosage form of a much smaller size compared to the currently marketed RINVOQ tablets.

Thus, the present disclosure provides solid dosage forms to enhance physical and chemical stability to, for example, improve appearance, dissolution, and/or decrease formation of degradation products; facilitate swallowability by providing tablets substantially less than 500 mg in size; provide substantially complete drug release; and/or provide a solid dosage form with improved or similar dissolution profile and/or bioavailability to that of RINVOQ.

In one embodiment, the solid dosage forms comprise upadacitinib or a pharmaceutically acceptable salt thereof, at least one pH-dependent polymer, and at least one release control material.

Without wishing to be bound by any particular theory, it is believed that as the solid dosage form comprising upadacitinib, a pH-dependent polymer, and a release control material begins to dissolve and erode upon administration and exposure to water in the low pH environment of the stomach, the pH-dependent polymer acts as a diffusion barrier, reducing drug release rate while the release control material hydrates, forming a viscous substance, a gel, and/or swells, thus together controlling the release rate of upadacitinib in the stomach. As the solid dosage form moves to the more basic pH environment of the intestine, the pH-dependent polymer then begins to dissolve and facilitates erosion of the release control material, allowing controlled release of upadacitinib in the intestine.

In one embodiment, the solid dosage forms comprise upadacitinib or a pharmaceutically acceptable salt thereof and a release rate modifier, wherein the release rate modifier is not a hygroscopic acidic pH modifier such as tartaric acid. In some such embodiments, the release rate modifier is an ion exchange resin. In some such embodiments, the release rate modifier is a non-acidic or a basic pH modifier and the solid dosage form optionally further comprises an anionic polymer or an anionic polysaccharide.

In one embodiment, the solid dosage forms comprise upadacitinib or a pharmaceutically acceptable salt thereof and a barrier layer covering a portion of the release surface of the solid dosage forms.

In one embodiment, the solid dosage forms comprise an osmotic pump drug release system.

In certain embodiments, upadacitinib or a pharmaceutically acceptable salt thereof is present in a solid dosage form, as described herein, in an amount sufficient to deliver between about 5 and about 50 mg, per unit dosage form, of upadacitinib free base equivalent. In some such embodiments, upadacitinib or a pharmaceutically acceptable salt thereof is present in a solid dosage form in an amount sufficient to deliver about 7.5 mg, per unit dosage form, of upadacitinib free base equivalent. In some such embodiments, upadacitinib or a pharmaceutically acceptable salt thereof is present in a solid dosage form in an amount sufficient to deliver about 15 mg, per unit dosage form, of upadacitinib free base equivalent. In some such embodiments, upadacitinib or a pharmaceutically acceptable salt thereof is present in a solid dosage form in an amount sufficient to deliver about 30 mg, per unit dosage form, of upadacitinib free base equivalent. In some such embodiments, upadacitinib or a pharmaceutically acceptable salt thereof is present in a solid dosage form in an amount sufficient to deliver about 45 mg, per unit dosage form, of upadacitinib free base equivalent.

The term “upadacitinib freebase” refers to freebase (non-salt, neutral) forms of upadacitinib. Examples of upadacitinib freebase solid state forms include amorphous upadacitinib freebase and crystalline freebases of upadacitinib. Specific examples of upadacitinib freebase solid state forms include but are not limited to Amorphous Upadacitinib Freebase, Upadacitinib Freebase Solvate Form A, Upadacitinib Freebase Hydrate Form B, Upadacitinib Freebase Hydrate Form C (which is a hemihydrate), and Upadacitinib Freebase Anhydrate Form D, each as described in International Applications WO 2017/066775 and WO 2018/165581, the contents of each of which are herein incorporated by reference.

A “pharmaceutically acceptable salt” of upadacitinib refers to those salts which are appropriate for use in a pharmaceutical composition and that are compatible with the solid dosage forms described herein. Such salts may be obtained, for example, by reaction of upadacitinib free base with inorganic acids such as hydrochloric acid, hydrobromic acid, sulfuric acid, nitric acid, and phosphoric acid or organic acids such as organic sulfonic acid, organic carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g., (+) or (−)-tartaric acid or mixtures thereof), amino acids (e.g., (+) or (−)-amino acids or mixtures thereof), and the like.

The term “upadacitinib freebase equivalent” refers to the amount of the neutral upadacitinib freebase (active ingredient) administered, free of any additional components in the solid state form, such as free of any solvent or water molecule(s) of a solvate or hydrate (including hemihydrate) solid state form, and free of any pharmaceutically acceptable salt counteranions of a pharmaceutically acceptable salt solid state form. For example, 15.4 mg of crystalline upadacitinib freebase hemihydrate (which includes ½ of a water molecule per upadacitinib freebase molecule) delivers 15 mg of upadacitinib freebase equivalent, while 30.7 mg of crystalline upadacitinib freebase hemihydrate (which includes ½ of a water molecule per upadacitinib freebase molecule) delivers 30 mg of upadacitinib freebase equivalent.

The term “anhydrate” as applied to a compound refers to a solid state wherein the compound contains no structural water within the crystal lattice.

The term “solid dosage form” is used interchangeably herein with pharmaceutical composition, and both refer to a solid formulation suitable for oral administration to a human. Exemplary solid dosage forms include, but are not limited to, tablets (coated or uncoated) and capsules. “Extended release” (also referred to as controlled or sustained release) solid dosage forms are formulated in such a manner as to slowly release the contained drug over an extended period of time, e.g., over a period of 0 to 20 hours, 0 to 18 hours, 0 to 16 hours, 0 to 14 hours, 0 to 12 hours, 0 to 10 hours, 0 to 8 hours, 0 to 6 hours, or 0 to 4 hours, e.g., wherein substantially complete release is attained from about 4 hours to about 20 hours, from about 4 hours to about 16 hours, from about 4 hours to about 10 hours, from about 4 hours to about 8 hours, from about 4 hours to about 6 hours, from about 6 hours to about 8 hours, from about 6 hours to about 10 hours, or from about 6 hours to about 12 hours, following ingestion (at time 0 hours). Compare, for example, immediate release solid dosage forms which permit the release of most or all of the active ingredient over a short period of time (e.g., typically around 60 minutes or less). In certain embodiments, the release is a substantially steady release of upadacitinib from the solid dosage form over the extended period of time. In certain embodiments, the release is a substantially complete release of upadacitinib from the solid dosage form over the extended period of time. In certain embodiments, the solid dosage form is an extended release tablet. As used herein “substantially steady” refers to a relatively constant rate of dissolution over the extended period of time. Furthermore, as used herein, “substantially complete” refers to at least 95% of upadacitinib released from the solid dosage form over the extended period of time. A complete release refers to 100% of upadacitinib being released from the solid dosage form over the extended period of time.

The term “pH-dependent polymer” refers to a polymer that is insoluble or only slightly soluble at a low pH (e.g., about pH 1 up to but less than pH 5) but becomes soluble at a higher pH (e.g., pH 5 and above). In certain embodiments, a pH-dependent polymer may become soluble at a pH range from about pH 5 and above, e.g., from about pH 5 to about pH 9, from about pH 5 to about pH 8, from about pH 5 to about pH 7, or from about pH 5 to about pH 6, which is generally less acidic than the gastric environment and roughly corresponds to pH values in the small intestine. Exemplary pH-dependent polymers include, but are not limited to, (i) enteric polymers, such as a hydroxyalkyl cellulose acetate succinate (e.g., hydroxypropylmethyl cellulose acetate succinate (HPMCAS)), hydroxyalkyl methyl cellulose phthalate (e.g., hydroxypropyl methyl cellulose phthalate (HPMCP)), cellulose acetate phthalate (CAP), polyvinylacetatephthalate (PVAP), a poly (meth) acrylate-methacrylic acid copolymer such as a methyl methacrylate-methacrylic acid copolymer (e.g., Eudragit® L 100 or Eudragit® S 100), and (ii) anionic polysaccharides, such as alginic acid, pectin, hyaluronic acid, carboxymethylcellulose, polyacrylic acid (PAA), and Pluronic-g-poly (acrylic acid) copolymers.

In certain embodiments, the pH-dependent polymer is selected from the group consisting of enteric polymers, anionic polysaccharides, and combinations thereof. In certain embodiments, the pH-dependent polymer is selected from the group consisting of hydroxyalkyl cellulose acetate succinate, hydroxyalkyl methyl cellulose phthalate, cellulose acetate phthalate, a poly (meth) acrylate-methacrylic acid copolymer, alginic acid, pectin, hyaluronic acid, carboxymethylcellulose, polyacrylic acid (PAA), Pluronic-g-poly (acrylic acid) copolymers, and combinations thereof. In certain embodiments, the pH-dependent polymer is selected from the group consisting of hydroxypropylmethylcellulose acetate succinate, alginic acid, and combinations thereof. In certain embodiments, the pH-dependent polymer is hydroxypropylmethylcellulose acetate succinate. In one embodiment, the pH-dependent polymer is hydroxypropyl methyl cellulose phthalate (HPMCP). In certain embodiments, the pH-dependent polymer is alginic acid.

In certain embodiments, the pH-dependent polymer is present in the solid dosage form in an amount sufficient to (a) provide substantially steady drug release between pH 1.1 and 6.8; (b) provide substantially complete drug release independent of tablet size, and particularly for tablets weighing less than 500 mg, such as from about 100 mg to about 400 mg; (c) control generation of upadacitinib degradation products to within pharmaceutically acceptable levels during the shelf-life of the solid dosage form; (d) provide a substantially similar dissolution profile compared to the RINVOQ extended release tablets; and/or (e) provide a consistent dissolution profile across the shelf-life of the solid dosage form.

In certain embodiments, the pH-dependent polymer is present in the solid dosage form in an amount from about 10% to about 40% by weight (w/w) of the solid dosage form. In certain embodiments, the pH-dependent polymer is present in the solid dosage form in an amount from about 15% to about 35% by weight (w/w) of the solid dosage form. In certain embodiments, the pH-dependent polymer is present in the solid dosage form in an amount from about 20% to about 30% by weight (w/w) of the solid dosage form. In some such embodiments, the solid dosage form comprises about 20% by weight (w/w) of the pH-dependent polymer. In other such embodiments, the solid dosage form comprises about 25% by weight (w/w) of the pH-dependent polymer. In still other such embodiments, the solid dosage form comprises about 30% by weight (w/w) of the pH-dependent polymer.

As used herein, a “release control material” is an excipient material whose primary function is to modify the duration of release of the active drug substance (upadacitinib) from the dosage form by, for example, swelling and/or forming a viscous substance or gel in water and/or at low pH. In certain embodiments, the release control material is a non-polymeric rate control material. For example, the non-polymeric rate control material may be a release control lipid, such as glyceryl dibehenate (e.g., Compritol®888). In other embodiments, the non-polymeric rate control material may include fatty acids, fatty acid esters, mono-, di-, and tri-glycerides of fatty acids, fatty alcohols, waxes of natural and synthetic origins with differing melting points, and hydrophobic polymers used in hydrophobic, non-swellable matrices. Examples include stearic acid, lauryl, cetyl or cetostearyl alcohol, glyceryl behenate, carnauba wax, beeswax, candelilla wax, microcrystalline wax and low molecular weight polyethylene. In other embodiments, the non-polymeric rate control material is an insoluble polymer. Insoluble polymers include fine powders of ammoniomethacrylate copolymers (Eudragit® RL100, PO, RS100, PO), polyvinyl acetate or its mixture with povidone (Kollidon® SR), ethyl cellulose (Ethocel®), cellulose acetate (CA-398-10), cellulose acetate butyrate (CAB-381-20), cellulose acetate propionate (CAP-482-20), and latex dispersions of insoluble polymers (Eudragit® NE-30D, RL-30D, RS-30D, Surelease®). In certain embodiments, the release control material is a release control polymer. In some such embodiments, the release control polymer is a hydrophilic polymer. Exemplary release control polymers include, but are not limited to, a cellulose derivative with a viscosity of between 100 and 100,000 mPA-s, hydroxypropylmethyl cellulose (e.g., Hypromellose 2208 or a controlled release grade of hydroxypropylmethyl cellulose, including the E, F, and K series), a copolymer of acrylic acid crosslinked with a polyalkenyl polyether (e.g., Carbopol® polymers), hydroxypropyl cellulose, hydroxyethyl cellulose, a non-ionic homopolymer of ethylene oxide (e.g., Polyox™), a water soluble natural gum of a polysaccharide (e.g., xanthan gum, alginate, locust bean gum, etc.), a crosslinked starch, polyvinyl acetate, and polyvinylpyrrolidone.

In certain embodiments, the at least one release control material is selected from the group consisting of hydroxypropylmethyl cellulose (HPMC), a copolymer of acrylic acid crosslinked with a polyalkenyl polyether, and combinations thereof. In certain embodiments, the at least one release control material is selected from the group consisting of hydroxypropylmethyl cellulose, hydroxyethyl cellulose, and combinations thereof. In certain embodiments, the at least one release control material is hydroxypropylmethyl cellulose (HPMC).

In certain embodiments, the release control material is present in the solid dosage form in an amount from about 10% to about 60% by weight (w/w) of the solid dosage form. In certain embodiments, the release control material is present in the solid dosage form in an amount from about 20% to about 50% by weight (w/w) of the solid dosage form.

In certain embodiments, the solid dosage form comprises low levels of a hygroscopic acidic pH modifier in the composition (e.g., less than 15%, less than 10%, less than 5%). In certain embodiments, the hygroscopic acidic pH modifier is a hygroscopic organic acid. The term “hygroscopic” is used adjectivally to refer to materials, such pharmaceutically acceptable excipients, that absorb or adsorb significant amounts of moisture from the air or surrounding atmosphere. When a “hygroscopic” material absorbs moisture from the air or surrounding atmosphere to the extent that said material undergoes gradual dissolution and/or liquefaction, the material is considered “deliquescent.” Deliquescence represents the most severe case of hygroscopicity. In certain embodiments, the solid dosage form comprising upadacitinib or a pharmaceutically acceptable salt thereof includes not more than 15% by weight (w/w) of a hydroscopic acidic pH modifier, not more than 10% by weight (w/w) of a hygroscopic acidic pH modifier, or not more than 5% by weight (w/w) of a hygroscopic acidic pH modifier. In certain embodiments, the solid dosage form comprising upadacitinib or a pharmaceutically acceptable salt thereof includes not more than 15% by weight (w/w) of a hygroscopic organic acid, not more than 10% by weight (w/w) of a hygroscopic organic acid, or not more than 5% by weight (w/w) of a hygroscopic organic acid. In certain embodiments, the hygroscopic organic acid is selected from the group consisting of tartaric acid, citric acid, and maleic acid. In certain further embodiments, the solid dosage form further comprises low amounts (e.g., less than 15%, 10%, 5%) of other hygroscopic pharmaceutically acceptable excipients or materials in the composition. In one embodiment, the solid dosage form includes at least one release rate modifier. In one embodiment, the at least one release rate modifier is selected from the group consisting of an ion exchange resin, a basic pH modifier, an acidic pH modifier, and combinations thereof. An ion exchange resin suitable for use as a release rate modifier is AmberLite™ IRP 69 or a resin having similar characteristics. In one embodiment, the at least one release rate modifier is AmberLite™ IRP 69. Basic pH modifiers suitable for use as a release rate modifier include, but are not limited to, sodium carbonate (Na₂CO₃), meglumine, tribasic sodium phosphate dodecahydrate (Na₃PO₄·12 H₂O), sodium hydroxide, sodium bicarbonate, magnesium oxide, potassium hydroxide, and calcium phosphate. In one embodiment, the at least one release rate modifier is sodium carbonate. In some such embodiments, the at least one release rate modifier is sodium carbonate monohydrate. Acidic pH modifiers suitable for use as a release rate modifier include, but are not limited to, fumaric acid. In one embodiment, the acidic pH modifier is not a hygroscopic acidic pH modifier. In one embodiment, the at least one release rate modifier is fumaric acid.

In one embodiment, the release rate modifier is present in the solid dosage form in an amount from about 5% to about 40% by weight (w/w) of the solid dosage form. In some such embodiments, the solid dosage form comprises an ion exchange resin and the ion exchange resin is present in the solid dosage form in an amount from about 20% to about 35% by weight (w/w) of the solid dosage form. In some such embodiments, the solid dosage form comprises about 30% by weight (w/w) of the ion exchange resin. In some such embodiments, the solid dosage form comprises a basic pH modifier and the basic pH modifier is present in the solid dosage form in an amount from about 5% to about 25% by weight (w/w) of the solid dosage form. In some such embodiments, the solid dosage form comprises about 10% by weight (w/w) of the basic pH modifier. In some such embodiments, the solid dosage form comprises an acidic pH modifier and the acidic pH modifier is present in the solid dosage form in an amount from about 10% to about 35% by weight (w/w) of the solid dosage form. In some such embodiments, the solid dosage form comprises about 25% by weight (w/w) of the acidic pH modifier. In other such embodiments, the solid dosage form comprises about 30% by weight (w/w) of the acidic pH modifier.

In certain embodiments, the solid dosage form includes additional pharmaceutically acceptable excipients (e.g., fillers, glidants, and/or lubricants), wherein the total amount of the additional pharmaceutically acceptable excipients is less than 50% by weight (w/w), less than 45% w/w, less than 40% w/w, less than 35% w/w, less than 30% w/w, less than 25% w/w, less than 20% w/w, less than 15% w/w, less than 10% w/w, or less than 5% w/w of the solid dosage form.

In certain embodiments, the solid dosage form comprises at least one excipient that functions as a filler. Fillers may include, for example, polyols, such as dextrose, isomalt, mannitol (such as spray dried mannitol (e.g., Pearlitol® 100SD, Pearlitol® 200SD)), sorbitol, lactose, and sucrose; natural or pre-gelatinized starch (such as potato starch, corn starch, Starch 1500®); microcrystalline cellulose (such as Avicel® PH 101 or Avicel® PH 102); lactose monohydrate (e.g., Foremost® 316 Fast Flo®); mixtures of isomaltulose derivatives (e.g., galenIQ™ 720); and combinations thereof.

In certain embodiments, the solid dosage form includes a filler selected from the group consisting of microcrystalline cellulose, lactose, mannitol, and combinations thereof. In some such embodiments, the filler is microcrystalline cellulose. In some such embodiments, the filler is lactose. In some such embodiments, the filler is mannitol.

In certain embodiments, one or more fillers are present in the solid dosage form in an amount from about 0.1% to about 50% by weight (w/w). In certain embodiments, the filler is present in the solid dosage form in an amount from about 15% to about 45% by weight (w/w).

In certain embodiments, the solid dosage form includes a first filler and a second filler, wherein the total amount of the first and second filler present in the solid dosage form is from about 15% to about 45% by weight (w/w). In some such embodiments, the first filler is microcrystalline cellulose. In some such embodiments, the second filler is mannitol.

In certain embodiments, the solid dosage form comprises at least one excipient that functions as a glidant. Glidants may include, for example, colloidal silicon dioxide, including highly dispersed silica (Aerosil®) or any other suitable glidant such as animal or vegetable fats or waxes.

In certain embodiments, a glidant is present in the solid dosage form in an amount from about 0.1% to about 5% by weight (w/w). In certain embodiments, a glidant is present in the solid dosage form in an amount from about 0.3% to about 2.5% by weight (w/w). In certain embodiments, a glidant is present in the solid dosage form in an amount from about 0.5% to about 1.5% by weight (w/w). In certain embodiments, the solid dosage form includes about 0.5% by weight (w/w) of a glidant. In certain embodiments, the solid dosage form includes about 1% by weight (w/w) of a glidant. In certain embodiments, the glidant is colloidal silicon dioxide.

In certain embodiments, the solid dosage form comprises at least one excipient that functions as a lubricant. Lubricants may include, for example, magnesium and calcium stearates, sodium stearyl fumarate, talc, or any other suitable lubricant.

In certain embodiments, a lubricant is present in the solid dosage form in an amount from about 0.1% to about 5% by weight (w/w). In certain embodiments, a lubricant is present in the solid dosage form in an amount from about 0.3% to about 2.5% by weight (w/w). In certain embodiments, a lubricant is present in the solid dosage form in an amount from about 0.5% to about 1.5% by weight (w/w). In certain embodiments, the solid dosage form includes about 1% by weight (w/w) of a lubricant. In certain embodiments, the lubricant is magnesium stearate. In certain embodiments, the lubricant is sodium stearyl fumarate.

As generally described herein, the present disclosure contemplates solid dosage forms comprising upadacitinib or a pharmaceutically acceptable salt thereof, at least one pH-dependent polymer, and at least one release control material.

In certain embodiments, the pH-dependent polymer is a component of a matrix system containing upadacitinib or a pharmaceutically acceptable salt thereof. In some such embodiments, the pH-dependent polymer is present in the solid dosage form matrix, but substantially absent from any coat surrounding the solid dosage form. While the pH-dependent polymer (e.g., an enteric polymer) is a component of the solid dosage form matrix, an enteric polymer may optionally and additionally be present as part of the film coat in order to allow for an even longer extended release. In certain embodiments, the solid dosage form does not comprise an enteric coat.

In certain embodiments, the release control material is a component of a matrix system containing upadacitinib or a pharmaceutically acceptable salt thereof.

In certain embodiments, the pH-dependent polymer is hydroxypropylmethylcellulose acetate succinate (HPMCAS) and the release control material is hydroxypropylmethyl cellulose (HPMC). In one embodiment, the pH-dependent polymer is hydroxypropylmethylcellulose phthalate (HPMCP) and the release control material is hydroxypropylmethyl cellulose (HPMC). In certain embodiments, the pH-dependent polymer is alginic acid and the release control material is hydroxypropylmethyl cellulose (HPMC). In some such embodiments, the pH-dependent polymer and the release control material are components of a matrix system containing upadacitinib or a pharmaceutically acceptable salt thereof.

In one embodiment, the solid dosage form comprises upadacitinib or a pharmaceutically acceptable salt thereof, at least one release control material, at least one pH dependent polymer, and is substantially free (e.g., greater than about 98%, 99%, 99.9% w/w) of a hygroscopic acidic pH modifier in the composition. In some such embodiments, the solid dosage form comprises upadacitinib or a pharmaceutically acceptable salt thereof, at least one release control material, at least one pH dependent polymer, and is substantially free of a hygroscopic organic acid. In one embodiment, the hygroscopic organic acid is selected from the group consisting of tartaric acid, citric acid, and maleic acid.

In one embodiment, the solid dosage form optionally comprises one or more additional pharmaceutically acceptable excipients. For example, the solid dosage form comprising upadacitinib or a pharmaceutically acceptable salt thereof, at least one release control material, and at least pH dependent polymer, may further optionally comprise one or more additional pharmaceutically acceptable excipients that function as fillers, binders, glidants and/or lubricants.

In certain embodiments, the solid dosage form comprises upadacitinib or a pharmaceutically acceptable salt thereof, hydroxypropylmethylcellulose acetate succinate (HPMC-AS) as a pH-dependent polymer, hydroxypropylmethyl cellulose (HPMC) as a release control material, and, optionally, at least one filler, at least one glidant, and/or at least one lubricant. In some such embodiments, the at least one filler is microcrystalline cellulose, lactose, mannitol, or a combination thereof. In some such embodiments, the at least one glidant is colloidal silicon dioxide. In some such embodiments, the at least one lubricant is sodium stearyl fumarate or magnesium stearate. In some such embodiments, the HPMC-AS and HPMC are components of a matrix system containing upadacitinib or a pharmaceutically acceptable salt thereof.

In one embodiment, the solid dosage form comprises upadacitinib or a pharmaceutically acceptable salt thereof, hydroxypropylmethylcellulose phthalate (HPMCP) as a pH-dependent polymer, hydroxypropylmethyl cellulose (HPMC) as a release control material, and, optionally, at least one filler, at least one glidant, and/or at least one lubricant. In some such embodiments, the at least one filler is microcrystalline cellulose, lactose, mannitol, or a combination thereof. In some such embodiments, the at least one glidant is colloidal silicon dioxide. In some such embodiments, the at least one lubricant is sodium stearyl fumarate or magnesium stearate. In some such embodiments, the HPMCP and HPMC are components of a matrix system containing upadacitinib or a pharmaceutically acceptable salt thereof.

In certain embodiments, the solid dosage form comprises upadacitinib or a pharmaceutically acceptable salt thereof, alginic acid as a pH-dependent polymer, hydroxypropylmethyl cellulose (HPMC) as a release control material, and, optionally, at least one filler, at least one glidant, and/or at least one lubricant. In some such embodiments, the at least one filler is microcrystalline cellulose, lactose, mannitol, or a combination thereof. In some such embodiments, the at least one glidant is colloidal silicon dioxide. In some such embodiments, the at least one lubricant is sodium stearyl fumarate or magnesium stearate. In some such embodiments, the alginic acid and HPMC are components of a matrix system containing upadacitinib or a pharmaceutically acceptable salt thereof.

In certain embodiments, the solid dosage form is a tablet, which may be coated with any suitable coating such as a film coat. A film coat may be used to, for example, contribute to the ease with which the tablet can be swallowed. A film coat may also be employed to improve taste and provide an elegant appearance. The film coat may comprise a polyvinyl alcohol-polyethylene glycol graft copolymer, such as Opadry®. The film coat may also comprise talc as an anti-adhesive. The film coat may account for less than about 5% by weight of the weight of the tablet.

As generally described herein, the present disclosure contemplates solid dosage forms comprising upadacitinib or a pharmaceutically acceptable salt thereof and at least one release rate modifier, such as an ion exchange resin.

In one embodiment, the release rate modifier is an ion exchange resin. In one embodiment, the ion exchange resin is a cation exchange resin. In one embodiment, the solid dosage forms comprise an upadacitinib-ion exchange resin complex. In some such embodiments, the upadacitinib-ion exchange resin complex comprises upadacitinib or a pharmaceutically acceptable salt thereof bound to an ion exchange resin.

Ion-exchange resins suitable for use in the solid dosage forms disclosed herein are water-insoluble and preferably comprise a pharmacologically inert organic and/or inorganic matrix containing functional groups that are ionic or capable of being ionized under appropriate conditions. In some such embodiments, the organic matrix is synthetic (e.g., a polymer or copolymer of acrylic acid, methacrylic acid, sulfonated styrene, sulfonated divinylbenzene). In some such embodiments, the inorganic matrix comprises silica gel modified by the addition of ionic groups.

Suitable ion exchange resins include, but are not limited to, a sulfonated copolymer comprising styrene and divinylbenzene. In some such embodiments, the moble, or exchangeable, cation is sodium. An exemplary cation ion exchange resin is AmberLite™ IRP 69 (DuPont).

In one embodiment, the solid dosage form comprises upadacitinib or a pharmaceutically acceptable salt thereof and at least one release rate modifier, and is substantially free (e.g., greater than about 98%, 99%, 99.9% w/w) of a hygroscopic acidic pH modifier in the composition. In some such embodiments, the solid dosage form comprises upadacitinib or a pharmaceutically acceptable salt thereof, at least one release control material, at least one release rate modifier, and is substantially free of a hygroscopic organic acid. In one embodiment, the hygroscopic organic acid is selected from the group consisting of tartaric acid, citric acid, and maleic acid.

In one embodiment, the solid dosage form optionally comprises one or more additional pharmaceutically acceptable excipients. For example, the solid dosage form comprising upadacitinib or a pharmaceutically acceptable salt thereof and at least one release rate modifier, may further optionally comprise one or more additional pharmaceutically acceptable excipients that function as fillers, binders, glidants and/or lubricants.

In one embodiment, the solid dosage form comprises upadacitinib or a pharmaceutically acceptable salt thereof, an ion exchange resin as a release rate modifier, and, optionally, at least one filler, at least one glidant, and/or at least one lubricant. In some such embodiments, the at least one filler is microcrystalline cellulose, lactose, mannitol, or a combination thereof. In some such embodiments, the at least one glidant is colloidal silicon dioxide. In some such embodiments, the at least one lubricant is sodium stearyl fumarate or magnesium stearate.

As generally described herein, the present disclosure contemplates solid dosage forms comprising upadacitinib or a pharmaceutically acceptable salt thereof, at least one release rate modifier, such as a basic pH modifier, and, optionally, at least one pH-dependent polymer.

In one embodiment, the release rate modifier is a basic pH modifier. Exemplary basic pH modifiers include, but are not limited to, sodium carbonate, meglumine, tribasic sodium phosphate dodecahydrate (Na₃PO₄·12 H₂O), sodium hydroxide, sodium bicarbonate, magnesium oxide, potassium hydroxide, and calcium phosphate. In some such embodiments, the release rate modifier is sodium carbonate monohydrate.

In one embodiment, the solid dosage form comprises upadacitinib or a pharmaceutically acceptable salt thereof, at least one basic pH modifier, and an anionic polymer or an anionic polysaccharide, such as hydroxypropyl methyl cellulose phthalate (HPMCP), cellulose acetate phthalate (CAP), hydroxypropyl methylcellulose acetate succinate (HPMCAS), polyvinylacetate phthalate (PVAP), methacrylic acid copolymers (Eudragit L), alginic acid, pectin, hyaluronic acid, or carboxymethylcellulose.

In one embodiment, the solid dosage form comprises upadacitinib or a pharmaceutically acceptable salt thereof, at least one basic pH modifier, and is substantially free (e.g., greater than about 98%, 99%, 99.9% w/w) of a hygroscopic acidic pH modifier in the composition. In some such embodiments, the solid dosage form comprises upadacitinib or a pharmaceutically acceptable salt thereof, at least one basic pH modifier, at least one pH dependent polymer, and is substantially free of a hygroscopic organic acid. In one embodiment, the hygroscopic organic acid is selected from the group consisting of tartaric acid, citric acid, and maleic acid.

In one embodiment, the solid dosage form optionally comprises one or more additional pharmaceutically acceptable excipients. For example, the solid dosage form comprising upadacitinib or a pharmaceutically acceptable salt thereof and at least one basic pH modifier, may further optionally comprise one or more additional pharmaceutically acceptable excipients that function as fillers, binders, glidants and/or lubricants. As another example, the solid dosage form comprising upadacitinib or a pharmaceutically acceptable salt thereof, at least one basic pH modifier, and at least one pH dependent polymer, may further optionally comprise one or more additional pharmaceutically acceptable excipients that function as fillers, binders, glidants and/or lubricants.

In one embodiment, the solid dosage form comprises upadacitinib or a pharmaceutically acceptable salt thereof, a basic pH modifier as a release rate modifier, and, optionally, at least one filler, at least one glidant, and/or at least one lubricant. In some such embodiments, the at least one filler is microcrystalline cellulose, lactose, mannitol, or a combination thereof. In some such embodiments, the at least one glidant is colloidal silicon dioxide. In some such embodiments, the at least one lubricant is sodium stearyl fumarate or magnesium stearate.

As generally described herein, the present disclosure contemplates solid dosage forms comprising upadacitinib or a pharmaceutically acceptable salt thereof and a barrier layer covering a portion of the release surface (e.g., a partially coated tablet).

In one embodiment, the solid dosage form comprises a barrier layer partially covering the release surface of the solid dosage form. In some such embodiments, the barrier layer is applied to a portion of the surface of the solid dosage form. For example, the barrier layer may be applied as a coating solution on one side of the solid dosage form. As another example, the barrier layer may be applied on one side of the solid dosage form by compression coating.

In one embodiment, the barrier layer comprises a pH-dependent polymer. In some such embodiments, the pH-dependent polymer is hydroxypropylmethyl cellulose acetate succinate (HPMCAS). For example, a film coating solution containing about 5% by weight HPMCAS may be applied to a portion (e.g., one side) of the solid dosage form. As another example, a compression coating layer containing about 92% by weight HPMCAS may be applied to a portion (e.g., one side) of the solid dosage form.

In one embodiment, the solid dosage form further comprises a release rate modifier. In one embodiment, the release rate modifier is an acidic pH modifier. In some such embodiments, the acidic pH modifier is not a hygroscopic pH modifier. In some such embodiments, the release rate modifier is fumaric acid.

In one embodiment, the solid dosage form comprises upadacitinib or a pharmaceutically acceptable salt thereof, at least one non-hygroscopic acidic pH modifier, a barrier layer comprising a pH-dependent polymer, and is substantially free (e.g., greater than about 98%, 99%, 99.9% w/w) of a hygroscopic acidic pH modifier in the composition. In one embodiment, the hygroscopic organic acid is selected from the group consisting of tartaric acid, citric acid, and maleic acid.

In one embodiment, the solid dosage form optionally comprises one or more additional pharmaceutically acceptable excipients. For example, the solid dosage form comprising upadacitinib or a pharmaceutically acceptable salt thereof, at least one acidic pH modifier, and a barrier layer comprising a pH-dependent polymer, may further optionally comprise one or more additional pharmaceutically acceptable excipients that function as fillers, binders, glidants and/or lubricants.

In one embodiment, the solid dosage form comprises upadacitinib or a pharmaceutically acceptable salt thereof, an acidic pH modifier as a release rate modifier, a barrier layer comprising a pH-dependent polymer, and, optionally, at least one filler, at least one glidant, and/or at least one lubricant. In some such embodiments, the at least one filler is microcrystalline cellulose, lactose, mannitol, or a combination thereof. In some such embodiments, the at least one glidant is colloidal silicon dioxide. In some such embodiments, the at least one lubricant is sodium stearyl fumarate or magnesium stearate.

As generally described herein, the present disclosure contemplates solid dosage forms comprising upadacitinib or a pharmaceutically acceptable salt thereof, wherein the solid dosage forms comprise an osmotic pump system.

In an exemplary osmotic pump system, a core is encased by a semi-permeable membrane having at least one drug delivery orifice. The core contains the active agent and, optionally, at least one osmogent. The semi-permeable membrane is permeable to aqueous fluids such as water or biological fluids, but impermeable to the active agent. When the system is exposed to an aqueous environment, water will penetrate through the semi-permeable membrane into the core. Osmotic pressure increases within the dosage form and the active agent (i.e., upadacitinib or a pharmaceutically acceptable salt thereof) is released through the drug delivery orifice.

Suitable osmogents include, but are not limited to, water soluble salts of inorganic acids (e.g., magnesium sulfate, magnesium chloride, sodium chloride, sodium sulfate, potassium chloride, sodium bicarbonate, sodium phosphate), osmotic polymers (e.g., polyoxyethylene, polyvinylpyrrolidone, polyacrylic acid, hydroxypropyl methylcellulose, hydroxyethylcellulose (HEC)), carbohydrates (e.g., raffinose, sucrose, glucose, sorbitol, xylitol), and combinations thereof. An exemplary osmogent is sorbitol, which is available as NEOSORB® P 60 W (Roquette).

Suitable materials for forming semi-permeable membranes include, but are not limited to, cellulose esters, cellulose monoesters, cellulose diesters, cellulose triesters, cellulose ethers, cellulose ester-ethers, and combinations thereof. In one embodiment, the semi-permeable membrane comprises cellulose acetate (CA). An exemplary semi-permeable membrane system is Opadry® CA Fully Formulated Osmotic Coating System (Colorcon).

In one embodiment, the core comprises more than one compartment or layer. For example the core may comprise a bi-layer tablet having an active agent-containing layer and a push layer. In one embodiment, the core comprises a separation layer between the active agent-containing layer and the push layer (e.g., a tri-layer tablet). In some such embodiments, the push layer comprises an osmotic polymer that facilitates swelling of the push layer upon exposure to an aqueous environment. Thus, when the osmotic pump system is exposed to an aqueous environment such as the gastrointestinal tract, the push layer swells and pushes the active agent through the drug delivery orifice.

In one embodiment, the osmotic polymer is a swellable hydrophilic polymer. Suitable osmotic polymers include, but are not limited to, polyoxyethylene, polyvinylpyrrolidone, polyacrylic acid, hydroxypropyl methylcellulose, hydroxyethylcellulose (HEC), and combinations thereof. An exemplary osmotic polymer is Natrosol™ 250HX (Ashland).

Osmotic pumps are well known in the art and have been described in the literature. For example, U.S. Pat. Nos. 4,088,864, 4,200,098, and 5,573,776 describe osmotic pumps and methods for their manufacture and are hereby incorporated by reference.

In general, an osmotic pump system can be formed by compressing a tablet of an osmotically active drug (or an osmotically inactive drug in combination with an osmogent) and then coating the tablet with a semi-permeable membrane. One or more drug delivery orifices may be drilled through the semi-permeable membrane. In one embodiment, the size of a drug delivery orifice is from about 0.1 mm to about 4.0 mm, such as, for example, about 0.5 mm, about 1.0 mm, about 1.5 mm, about 2.0 mm, or about 2.5 mm. Alternatively, orifice(s) through the wall may be formed in situ by incorporating leachable pore forming materials in the semi-permeable membrane. In operation, the exterior aqueous based fluid is imbibed through the semi-permeable membrane and contacts with at least one active agent to form a solution or suspension of the active agent. The active agent solution or suspension is then “pumped” out through the orifice as fresh fluid is imbibed through the semi-permeable membrane.

In one embodiment, the solid dosage form comprises (i) a core comprising an active agent-containing layer comprising upadacitinib or a pharmaceutically acceptable salt thereof and an osmogent and a push layer comprising an osmotic polymer such as hydroxyethylcellulose (HEC) and (ii) a semi-permeable membrane surrounding the core. In some such embodiments, the semi-permeable membrane contains at least one drug delivery orifice. In some such embodiments, the at least one drug delivery orifice is mechanically or laser drilled into the semi-permeable membrane.

In one embodiment, the solid dosage form optionally comprises one or more additional pharmaceutically acceptable excipients. For example, the core of the solid dosage form comprising upadacitinib or a pharmaceutically acceptable salt thereof, an osmogent, and an osmotic polymer may further optionally comprise one or more additional pharmaceutically acceptable excipients that function as fillers, binders, glidants and/or lubricants. In some such embodiments, the solid dosage for further optionally comprises a lubricant such as magnesium stearate.

In at least one embodiment, this disclosure is directed to providing upadacitinib or a pharmaceutically acceptable salt thereof in a single, stable oral dosage form. The solid dosage forms disclosed herein are intended for pharmaceutical use in human subjects. Accordingly, they should be of an appropriate size and weight for oral human administration (e.g., they should have a total weight of less than 500 mg, and, preferably from about 100 mg to about 400 mg, more preferably from about 150 to about 300 mg). In certain embodiments, the solid dosage form is less than 400 mg, less than 350 mg, less than 300 mg, less than 250 mg, less than 200 mg, less than 150 mg in total weight. In some such embodiments, the solid dosage form is from about 150 mg to about 300 mg in total weight, such as about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg in total weight. In order to facilitate the intake of such a dosage form by a mammal, the dosage form may be shaped into an appropriate shape such as a round or ovaloid or elongated shape.

In at least one embodiment, the solid dosage form is stable during, for example, storage, distribution, and the duration of the product's shelf-life (e.g., up to two years at room temperature/ambient conditions).

In certain embodiments, the dissolution profile of a stable solid dosage form does not materially change over time.

In certain embodiments, a stable solid dosage form exhibits less degradation of upadacitinib or a pharmaceutically acceptable salt thereof and/or lower amounts of degradation products over time compared to RINVOQ.

Solid dosage forms may be assessed for stability following storage for at least two weeks, at least one month, at least two months, at least three months, at least six months, at least nine months, at least twelve months, at least eighteen months, at least twenty four months, at least thirty months, or at least thirty six months. In particular, storage stability may be assessed at time intervals of one, three, six, nine, twelve, eighteen, twenty four, thirty, thirty six, and/or forty eight months. Storage conditions may be long term, intermediate, or accelerated conditions. In particular, storage conditions may be, for example, 25° C.±2° C./40% relative humidity (RH)±5% RH, 25° C.±2° C./60% RH±5% RH, 30° C.±2° C./35% RH±5% RH, 30° C.±2° C./65% RH±5% RH, 30° C.±2° C./75% RH±5% RH, 40° C.±2° C./25% RH±5% RH, 40° C.±2° C./50% RH±5% RH, 40° C.±2° C./75% RH±5% RH, 50° C.±2° C./75% RH±5% RH, 60° C.±2° C./5% RH±5% RH, 60° C.±2° C./40% RH±5% RH, 60° C.±2° C./50% RH±5% RH, 70° C.±2° C./5% RH±5% RH, 70° C.±2° C./75% RH±5% RH, 80° C.±2° C./40% RH ±5% RH, and/or 80° C.±2° C./75% RH±5% RH.

In certain embodiments, storage of the solid dosage form is at 25° C. +2° C. and 60% +5% relative humidity for between about 3 months and about 48 months, between about 6 months and about 36 months, or between about 12 months and about 24 months.

In certain embodiments, the stable solid dosage form comprises no more than pharmaceutically acceptable levels of an upadacitinib degradation product. In some such embodiments, the excipients contained in the solid dosage form control generation of upadacitinib degradation products to within pharmaceutically acceptable levels during the shelf-life of the solid dosage form.

One exemplary degradation product of upadacitinib is (3S,4R)-3-ethyl-4-(3-(hydroxymethyl)-3H-imidazo[1,2-a]pyrrolo[2,3-e]pyrazin-8-yl)-N-(2,2,2-trifluoroethyl) pyrrolidine-1-carboxamide (upadacitinib hydroxymethyl impurity; “UHM impurity”), depicted in FIG. 2. In certain embodiments, the solid dosage form comprises no more than 1% of the UHM impurity. In certain embodiments, the solid dosage form comprises no more than 0.5% of the UHM impurity. In certain embodiments, the solid dosage form comprises no more than 0.2% of the UHM impurity.

In certain embodiments, the solid dosage form comprises no more than 0.2% of the UHM impurity at product release and no more than 0.5% of the UHM impurity at the end of the dosage form's shelf life.

In certain embodiments, the UHM impurity is present in a solid dosage form in an amount less than 0.5% by weight after storage for at least one month, at least two months, at least six months, at least nine months, at least twelve months, at least eighteen months, at least twenty-four months, at least thirty months, or at least thirty-six months at long term, intermediate, or accelerated conditions. In some such embodiments, storage conditions may be 25° C.±2° C./60% RH±5% RH. In some such embodiments, storage conditions may be 40° C.±2° C./75% RH±5% RH.

In certain embodiments, the solid dosage form comprises no more than 2.5% water content at release and no more than 4.0% water content at the end of the dosage form's shelf life.

In certain embodiments, the solid dosage form exhibits a post-storage dissolution profile that is substantially similar to an initial dissolution profile of the solid dosage form (e.g., prior to storage).

Assay and degradation product determination of solid dosage forms, and more particularly tablets, may be performed using methods and equipment familiar to those skilled in the art, e.g., with HPLC with UV detection. In certain embodiments, dissolution is assessed utilizing USP apparatus I (basket) at a rotation speed of 150 rpm in 900 mL of pH 6.8, 0.025 M sodium phosphate buffer containing 2.75% sodium chloride at 37° C.±0.5° C. In certain embodiments, dissolution is assessed utilizing USP apparatus I (basket) at a rotation speed of 150 rpm in 900 mL of pH 6.8, 0.025 M sodium phosphate buffer at 37° C.±0.5° C. In certain embodiments, dissolution is assessed utilizing USP apparatus I (basket) at a rotation speed of 150 rpm in 900 mL of pH 6.8, 0.050 M sodium phosphate buffer at 37° C.±0.5° C. In certain embodiments, dissolution is assessed utilizing USP apparatus I (basket) at a rotation speed of 150 rpm in 900 mL of pH 1.1, 0.1 N HCl at 37° C.±0.5° C.

In certain embodiments, the solid dosage form, when added to a test medium in a standard USP basket apparatus with a rotation speed of 150 rpm, shows drug release for at least 4 hours, at least 6 hours, or at least 8 hours. In certain embodiments, the release is approximately linear release, showing substantially similar amount of drug release per unit time, over at least 4 hours, at least 6 hours, or at least 8 hours.

In certain embodiments, the solid dosage form, when added to a test medium in a standard USP basket apparatus with a rotation speed of 150 rpm, dissolves not more than 85% of the solid state form of upadacitinib after passage of about 1 hour; not more than 85% of the solid state form of upadacitinib after passage of about 2 hours; from about 10% to about 65% of the solid state form of upadacitinib after passage of about 2 hours; from about 35% to about 90% of the solid state form of upadacitinib after passage of about 4 hours, and/or from about 70% to 100% of the solid state form of upadacitinib after passage of about 10 hours. In some such embodiments, the test medium comprises 900 mL of pH 6.8, 0.025 M sodium phosphate buffer containing 2.75% sodium chloride at 37° C.±0.5° C. In some such embodiments, the test medium comprises 900 mL of pH 6.8, 0.025 M sodium phosphate buffer at 37° C.±0.5° C. In some such embodiments, the test medium comprises 900 mL of pH 6.8, 0.050 M sodium phosphate buffer at 37° C.±0.5° C. In some such embodiments, the test medium comprises 900 mL of pH 1.1, 0.1 N HCl at 37° C.±0.5° C.

In certain embodiments, the solid dosage form, when added to a test medium comprising 900 mL of pH 6.8, sodium phosphate buffer at 37° C.±0.5° C. in a standard USP basket apparatus with a rotation speed of 150 rpm, dissolves not more than about 80% of the solid state form of upadacitinib after passage of about 4 hours and/or from about 80% to 100% of the solid state form of upadacitinib after passage of about 10 hours.

Dissolution profiles can be compared using model independent or model dependent methods. A model independent approach using a similarity factor, and comparison criteria are described in SUPAC-MR, Modified Release Solid dosage forms (September 1997).

Dissolution profiles may be compared using the following equation that defines a similarity factor (f₂):

$${f}_{2} = 50 log \{ [ 1 + 1 / n {\sum }_{t = 1}^{n} {( {R}_{t} - {T}_{t} )}^{2} ] \times 100 \}$$

where log=logarithm to base 10, n=number of sampling time points, Σ=summation over all time points, Rₜ=dissolution at time point t of the reference (e.g., initial assessment), Tₜ=dissolution at time point t of the test (e.g., post-storage assessment).

An f₂ value between 50 and 100 suggests the two dissolution profiles are similar. Also, in certain embodiments, the average difference at any dissolution sampling time point should be not greater than about 25%, alternatively not greater than about 15%, or alternatively not greater than about 10% between the post-storage and initial dissolution profiles.

In certain embodiments, the solid dosage form exhibits a dissolution profile that is similar to a dissolution profile of the formulations for marketed (or to-be-marketed) RINVOQ extended release tablets as set forth herein. Thus, in certain embodiments, the average difference at any dissolution sampling time point should be not greater than about 25%, alternatively not greater than about 15%, or alternatively not greater than about 10% between the solid dosage form dissolution profile and the RINVOQ dissolution profile.

In certain embodiments, the percentage of compound released from the solid dosage form at any dissolution sampling time point is within about 25%, alternatively within about 15%, or alternatively within about 10% of the percentage of compound released from a marketed (or to-be-marketed) RINVOQ extended release tablet. For example, a reference sample (e.g., RINVOQ) in which about 91% and about 100% of upadacitinib free base equivalent was released after 6 and 8 hours, respectively, using USP I method at a rotation speed of 150 rpm in 900 ml at 37° C. of pH 1.1, 0.1N HCl, a test sample (e.g., a solid dosage form described herein) would be considered to have a similar dissolution profile if from about 68% to about 100% and/or from about 75% to about 100% of upadacitinib free base equivalent was released after 6 and 8 hours, respectively, under the same conditions. In some such embodiments, a reference sample (e.g., RINVOQ) in which about 75%, about 91%, and about 100% of upadacitinib free base equivalent was released after 4, 6, and 8 hours, respectively, using USP I method at a rotation speed of 150 rpm in 900 ml at 37° C. of pH 1.1, 0.1N HCl, a test sample (e.g., a solid dosage form described herein) would be considered to have a similar dissolution profile if from about 56% to about 94%, from about 68% to about 100%, and/or from about 75% to about 100% of upadacitinib free base equivalent was released after 4, 6, and 8 hours, respectively, under the same conditions.

As another example, a reference sample (e.g., RINVOQ) in which about 68% and about 79% of upadacitinib free base equivalent was released after 6 and 8 hours, respectively, using USP I method at a rotation speed of 150 rpm in 900 ml at 37° C. of pH 6.8, 0.025 M sodium phosphate buffer containing 2.75% sodium chloride, a test sample (e.g., a solid dosage form described herein) would be considered to have a similar dissolution profile if from about 51% to about 85% and/or from about 59% to about 99% of upadacitinib free base equivalent was released after 6 and 8 hours, respectively, under the same conditions. In some such embodiments, a reference sample (e.g., RINVOQ) in which about 54%, about 68%, and about 79% of upadacitinib free base equivalent was released after 4, 6, and 8 hours, respectively, using USP I method at a rotation speed of 150 rpm in 900 ml at 37° C. of pH 6.8, 0.025 M sodium phosphate buffer containing 2.75% sodium chloride, a test sample (e.g., a solid dosage form described herein) would be considered to have a similar dissolution profile if from about 41% to about 68%, from about 51% to about 85%, and/or from about 59% to about 99% of upadacitinib free base equivalent was released after 4, 6, and 8 hours, respectively, under the same conditions. In some such embodiments, a reference sample (e.g., RINVOQ) in which about 54%, about 68%, about 79%, and about 86% of upadacitinib free base equivalent was released after 4, 6, 8, and 10 hours, respectively, using USP I method at a rotation speed of 150 rpm in 900 ml at 37° C. of pH 6.8, 0.025 M sodium phosphate buffer containing 2.75% sodium chloride, a test sample (e.g., a solid dosage form described herein) would be considered to have a similar dissolution profile if from about 41% to about 68%, from about 51% to about 85%, from about 59% to about 99%, and/or from about 65% to about 100% of upadacitinib free base equivalent was released after 4, 6, 8, and 10 hours, respectively, under the same conditions. In some such embodiments, a reference sample (e.g., RINVOQ) in which about 54%, about 68%, about 79%, about 86%, and about 90% of upadacitinib free base equivalent was released after 4, 6, 8, 10, and 12 hours, respectively, using USP I method at a rotation speed of 150 rpm in 900 ml at 37° C. of pH 6.8, 0.025 M sodium phosphate buffer containing 2.75% sodium chloride, a test sample (e.g., a solid dosage form described herein) would be considered to have a similar dissolution profile if from about 41% to about 68%, from about 51% to about 85%, from about 59% to about 99%, from about 65% to about 100%, and/or from about 68% to about 100% of upadacitinib free base equivalent was released after 4, 6, 8, 10, and 12 hours, respectively, under the same conditions. In some such embodiments, a reference sample (e.g., RINVOQ) in which about 54%, about 68%, about 79%, about 86%, about 90%, and about 95% of upadacitinib free base equivalent was released after 4, 6, 8, 10, 12, and 16 hours, respectively, using USP I method at a rotation speed of 150 rpm in 900 ml at 37° C. of pH 6.8, 0.025 M sodium phosphate buffer containing 2.75% sodium chloride, a test sample (e.g., a solid dosage form described herein) would be considered to have a similar dissolution profile if from about 41% to about 68%, from about 51% to about 85%, from about 59% to about 99%, from about 65% to about 100%, from about 68% to about 100%, and/or from about 71% to about 100% of upadacitinib free base equivalent was released after 4, 6, 8, 10, 12, and 16 hours, respectively, under the same conditions. In some such embodiments, a reference sample (e.g., RINVOQ) in which about 54%, about 68%, about 79%, about 86%, about 90%, about 95%, and about 97% of upadacitinib free base equivalent was released after 4, 6, 8, 10, 12, 16, and 18 hours, respectively, using USP I method at a rotation speed of 150 rpm in 900 ml at 37° C. of pH 6.8, 0.025 M sodium phosphate buffer containing 2.75% sodium chloride, a test sample (e.g., a solid dosage form described herein) would be considered to have a similar dissolution profile if from about 41% to about 68%, from about 51% to about 85%, from about 59% to about 99%, from about 65% to about 100%, from about 68% to about 100%, from about 71% to about 100%, and/or from about 73% to about 100% of upadacitinib free base equivalent was released after 4, 6, 8, 10, 12, 16, and 18 hours, respectively, under the same conditions. In some such embodiments, a reference sample (e.g., RINVOQ) in which about 54%, about 68%, about 79%, about 86%, about 90%, about 95%, about 97%, and about 98% of upadacitinib free base equivalent was released after 4, 6, 8, 10, 12, 16, 18, and 20 hours, respectively, using USP I method at a rotation speed of 150 rpm in 900 ml at 37° C. of pH 6.8, 0.025 M sodium phosphate buffer containing 2.75% sodium chloride, a test sample (e.g., a solid dosage form described herein) would be considered to have a similar dissolution profile if from about 41% to about 68%, from about 51% to about 85%, from about 59% to about 99%, from about 65% to about 100%, from about 68% to about 100%, from about 71% to about 100%, from about 73% to about 100%, and/or from about 74% to about 100% of upadacitinib free base equivalent was released after 4, 6, 8, 10, 12, 16, 18, and 20 hours, respectively, under the same conditions.

As yet another example, a reference sample (e.g., RINVOQ) in which about 80% and about 89% of upadacitinib free base equivalent was released after 6 and 8 hours, respectively, using USP I method at a rotation speed of 150 rpm in 900 ml at 37° C. of pH 6.8, 0.025 M sodium phosphate buffer, a test sample (e.g., a solid dosage form described herein) would be considered to have a similar dissolution profile if from about 60% to about 100% and/or from about 67% to about 100% of upadacitinib free base equivalent was released after 6 and 8 hours, respectively, under the same conditions. In some such embodiments, a reference sample (e.g., RINVOQ) in which about 65%, about 80%, and about 89% of upadacitinib free base equivalent was released after 4, 6, and 8 hours, respectively, using USP I method at a rotation speed of 150 rpm in 900 ml at 37° C. of pH 6.8, 0.025 M sodium phosphate buffer, a test sample (e.g., a solid dosage form described herein) would be considered to have a similar dissolution profile if from about 49% to about 81%, from about 60% to about 100%, and/or from about 67% to about 100% of upadacitinib free base equivalent was released after 4, 6, and 8 hours, respectively, under the same conditions. In some such embodiments, a reference sample (e.g., RINVOQ) in which about 65%, about 80%, about 89%, and about 94% of upadacitinib free base equivalent was released after 4, 6, 8, and 10 hours, respectively, using USP I method at a rotation speed of 150 rpm in 900 ml at 37° C. of pH 6.8, 0.025 M sodium phosphate buffer, a test sample (e.g., a solid dosage form described herein) would be considered to have a similar dissolution profile if from about 49% to about 81%, from about 60% to about 100%, from about 67% to about 100%, and/or from about 71% to about 100% of upadacitinib free base equivalent was released after 4, 6, 8, and 10 hours, respectively, under the same conditions. In some such embodiments, a reference sample (e.g., RINVOQ) in which about 65%, about 80%, about 89%, about 94%, and about 97% of upadacitinib free base equivalent was released after 4, 6, 8, 10, and 12 hours, respectively, using USP I method at a rotation speed of 150 rpm in 900 ml at 37° C. of pH 6.8, 0.025 M sodium phosphate buffer, a test sample (e.g., a solid dosage form described herein) would be considered to have a similar dissolution profile if from about 49% to about 81%, from about 60% to about 100%, from about 67% to about 100%, from about 71% to about 100%, and/or from about 73% to about 100% of upadacitinib free base equivalent was released after 4, 6, 8, 10, and 12 hours, respectively, under the same conditions. In some such embodiments, a reference sample (e.g., RINVOQ) in which about 65%, about 80%, about 89%, about 94%, about 97%, and about 100% of upadacitinib free base equivalent was released after 4, 6, 8, 10, 12, and 16 hours, respectively, using USP I method at a rotation speed of 150 rpm in 900 ml at 37° C. of pH 6.8, 0.025 M sodium phosphate buffer, a test sample (e.g., a solid dosage form described herein) would be considered to have a similar dissolution profile if from about 49% to about 81%, from about 60% to about 100%, from about 67% to about 100%, from about 71% to about 100%, from about 73% to about 100%, and/or from about 75% to about 100% of upadacitinib free base equivalent was released after 4, 6, 8, 10, 12, and 16 hours, respectively, under the same conditions.

As still another example, a reference sample (e.g., RINVOQ) in which about 77% and about 87% of upadacitinib free base equivalent was released after 6 and 8 hours, respectively, using USP I method at a rotation speed of 150 rpm in 900 ml at 37° C. of pH 6.8, 0.050 M sodium phosphate buffer, a test sample (e.g., a solid dosage form described herein) would be considered to have a similar dissolution profile if from about 58% to about 96% and/or from about 65% to about 100% of upadacitinib free base equivalent was released after 6 and 8 hours, respectively, under the same conditions. In some such embodiments, a reference sample (e.g., RINVOQ) in which about 63%, about 77%, and about 87% of upadacitinib free base equivalent was released after 4, 6, and 8 hours, respectively, using USP I method at a rotation speed of 150 rpm in 900 ml at 37° C. of pH 6.8, 0.050 M sodium phosphate buffer, a test sample (e.g., a solid dosage form described herein) would be considered to have a similar dissolution profile if from about 47% to about 79%, from about 58% to about 96%, and/or from about 65% to about 100% of upadacitinib free base equivalent was released after 4, 6, and 8 hours, respectively, under the same conditions. In some such embodiments, a reference sample (e.g., RINVOQ) in which about 63%, about 77%, about 87%, and about 93% of upadacitinib free base equivalent was released after 4, 6, 8, and 10 hours, respectively, using USP I method at a rotation speed of 150 rpm in 900 ml at 37° C. of pH 6.8, 0.050 M sodium phosphate buffer, a test sample (e.g., a solid dosage form described herein) would be considered to have a similar dissolution profile if from about 47% to about 79%, from about 58% to about 96%, from about 65% to about 100%, and/or from about 70% to about 100% of upadacitinib free base equivalent was released after 4, 6, 8, and 10 hours, respectively, under the same conditions. In some such embodiments, a reference sample (e.g., RINVOQ) in which about 63%, about 77%, about 87%, about 93%, and about 96% of upadacitinib free base equivalent was released after 4, 6, 8, 10, and 12 hours, respectively, using USP I method at a rotation speed of 150 rpm in 900 ml at 37° C. of pH 6.8, 0.050 M sodium phosphate buffer, a test sample (e.g., a solid dosage form described herein) would be considered to have a similar dissolution profile if from about 47% to about 79%, from about 58% to about 96%, from about 65% to about 100%, from about 70% to about 100%, and/or from about 72% to about 100% of upadacitinib free base equivalent was released after 4, 6, 8, 10, and 12 hours, respectively, under the same conditions. In some such embodiments, a reference sample (e.g., RINVOQ) in which about 63%, about 77%, about 87%, about 93%, about 96%, and about 100% of upadacitinib free base equivalent was released after 4, 6, 8, 10, 12, and 16 hours, respectively, using USP I method at a rotation speed of 150 rpm in 900 ml at 37° C. of pH 6.8, 0.050 M sodium phosphate buffer, a test sample (e.g., a solid dosage form described herein) would be considered to have a similar dissolution profile if from about 47% to about 79%, from about 58% to about 96%, from about 65% to about 100%, from about 70% to about 100%, from about 72% to about 100%, and/or from about 75% to about 100% of upadacitinib free base equivalent was released after 4, 6, 8, 10, 12, and 16 hours, respectively, under the same conditions.

In certain embodiments, a solid dosage form described herein has a dissolution profile similar to a marketed (or to-be-marketed) RINVOQ extended release tablet at 6 hours. In certain embodiments, a solid dosage form described herein has a dissolution profile similar to a marketed (or to-be-marketed) RINVOQ extended release tablet at 8 hours. In certain embodiments, a solid dosage form described herein has a dissolution profile similar to a marketed (or to-be-marketed) RINVOQ extended release tablet at 6 and 8 hours. In certain embodiments, a solid dosage form described herein has a dissolution profile similar to a marketed (or to-be-marketed) RINVOQ extended release tablet at 4, 6, and 8 hours. In certain embodiments, a solid dosage form described herein has a dissolution profile similar to a marketed (or to-be-marketed) RINVOQ extended release tablet at 6, 8, and 10 hours. In certain embodiments, a solid dosage form described herein has a dissolution profile similar to a marketed (or to-be-marketed) RINVOQ extended release tablet at 6, 8, 10, and 12 hours. In certain embodiments, a solid dosage form described herein has a dissolution profile similar to a marketed (or to-be-marketed) RINVOQ extended release tablet at 6, 8, 10, 12, and 16 hours. In certain embodiments, a solid dosage form described herein has a dissolution profile similar to a marketed (or to-be-marketed) RINVOQ extended release tablet at 4, 6, 8, 10, 12, and 16 hours.

The solid dosage form may be prepared by any suitable method. Methods such as direct compression, dry granulation, and wet or melt granulation may be used to blend upadacitinib or a pharmaceutically acceptable salt thereof with one or more excipients.

In certain embodiments, the solid dosage form comprises a tablet. In some such embodiments, the tablet is a compressed and/or milled tablet. For example, in some embodiments, the tablet is formed by blending the components (e.g., including the active ingredient and at least one pharmaceutically acceptable carrier). The components can then be either directly compressed, or one or more of the components can be granulated prior to compression. In one embodiment, milling is performed using a mill fitted with any suitable size screen (e.g., a fitted with a screen size of from about 600 to about 1400 μm or about 610 μm or about 1397 μm). Compression can be done in a tablet press, such as in a steel die between two moving punches.

In other embodiments, the compressed and/or milled tablet is formulated using a wet granulation process. Use of wet granulation helps reduce and/or eliminate sticking that may occur when compression without wet granulation (e.g., direct compression) is used to formulate the tablets.

### EXEMPLARY EMBODIMENTS

Embodiment 1. In one embodiment, the extended release solid form comprises upadacitinib, or a pharmaceutically acceptable salt thereof, at least one pH dependent polymer and at least one release control material.

Embodiment 2. The extended release solid dosage form of Embodiment 1, wherein the dosage form comprises less than 10% by weight of a hygroscopic acidic pH modifier.

Embodiment 3. The extended release solid dosage form of Embodiment 1, wherein the dosage form is substantially free of a hygroscopic acidic pH modifier.

Embodiment 4. The extended release solid dosage form of any of Embodiments 2-3, wherein the hygroscopic acidic pH modifier is an organic acid.

Embodiment 5. The extended release solid dosage form of Embodiment 4, wherein the hygroscopic organic acid is selected from the group consisting of tartaric acid, citric acid, and maleic acid.

Embodiment 6. The extended release solid dosage form of any one of Embodiments 1-5, wherein the solid dosage form further comprises one or more additional excipients selected from the group consisting of a filler, a binder, a glidant, a lubricant, a film coat, and combinations thereof; wherein the one or more additional excipients are present in an amount less than 50% w/w of the solid dosage form.

Embodiment 7. The extended release solid dosage form of any one of Embodiments 1-6, wherein the at least one pH dependent polymer and the at least one release control material comprise a matrix system containing upadacitinib or a pharmaceutically acceptable salt thereof.

Embodiment 8. The extended release solid dosage form of any one of Embodiments 1-7, wherein the dosage form does not comprise an enteric film coat.

Embodiment 9. The extended release solid dosage form of any one of Embodiments 1-8, wherein the at least one pH dependent polymer is an enteric polymer or an anionic polysaccharide.

Embodiment 10. The extended release solid dosage form of any one of Embodiments 1-9, wherein the at least one pH dependent polymer is hydroxypropylmethyl cellulose acetate succinate (HPMCAS) or alginic acid.

Embodiment 11. The extended release solid dosage form of any one of Embodiments 1-10, wherein the at least one pH dependent polymer is present in an amount from about 20% to about 40% w/w and the at least one release control material is present in an amount from about 30% to about 60% w/w of the solid dosage form.

Embodiment 12. The extended release solid dosage form of any one of Embodiments 1-11, wherein the at least one release control material is a release control polymer.

Embodiment 13. The extended release solid dosage form of any one of Embodiments 1-11, wherein the at least one release control polymer is selected from the group consisting of hydroxypropylmethyl cellulose (HPMC), hydroxypropyl cellulose, hydroxyethyl cellulose, a copolymer of acrylic acid crosslinked with a polyalkenyl polyether (Carbopol), non-ionic homopolymer of ethylene oxide (Polyox), a water soluble natural gum of a polysaccharide, crosslinked starch, polyvinyl acetate, polyvinylpyrrolidone, and combinations thereof.

Embodiment 14. The extended release solid dosage form of any one of Embodiments 1-13, wherein the upadacitinib or pharmaceutically acceptable salt thereof is present in the solid dosage form in an amount sufficient to deliver 5 mg to 50 mg, per unit dosage form, of upadacitinib free base equivalent.

Embodiment 15. The extended release solid dosage form of any one of Embodiments 1-14, wherein after storage the solid dosage form continues to retain (a) pharmaceutically acceptable levels of an upadacitinib degradation product and/or (b) a post-storage dissolution profile that is substantially similar to an initial dissolution profile.

Embodiment 16. The extended release solid dosage form of Embodiment 15, wherein the solid dosage form comprises no more than 0.5% w/w of the upadacitinib degradation product during a shelf-life of the solid dosage form, wherein the shelf-life of the solid dosage form is about 6 months, about 12 months, about 18 months, about 24 months, about 30 months, or about 36 months.

Embodiment 17. The extended release solid dosage form of Embodiment 15 or Embodiment 16, wherein the upadacitinib degradation product is (3S,4R)-3-ethyl-4-(3-(hydroxymethyl)-3H-imidazo[1,2-a]pyrrolo[2,3-e]pyrazin-8-yl)-N-(2,2,2-trifluoroethyl) pyrrolidine-1-carboxamide.

Embodiment 18. The extended release solid dosage form of any one of Embodiments 15-17, wherein storage of the solid dosage form is at 25° C.±2° C. and 60%±5% relative humidity for between about 3 months and about 48 months, about 6 months and about 36 months, or about 12 months and about 24 months.

Embodiment 19. The extended release solid dosage form of any one of Embodiments 15-18, wherein the initial dissolution profile is not more than about 85% of upadacitinib released from the solid dosage form in about 1 or about 2 hours, from about 10% to about 65% of upadacitinib released from the solid dosage form in about 2 hours, from about 35% to about 90% of upadacitinib released from the solid dosage form in about 4 hours, and/or from about 70% to 100% of upadacitinib released from the solid dosage form in about 10 hours; and wherein the initial dissolution profile is measured at 37° C.±2° C. using USP I apparatus at a rotation speed of 150 rpm in 900 mL of (1) pH 6.8, 0.025 M sodium phosphate buffer containing 2.75% sodium chloride, (2) pH 6.8, 0.025 M phosphate buffer, (3) pH 6.8, 0.050 M phosphate buffer, or (4) pH 1.1, 0.1N HCl.

Embodiment 20. The extended release solid dosage form of any one of Embodiments 15-19, wherein the at least one pH dependent polymer is an enteric polymer or an anionic polysaccharide.

Embodiment 21. The extended release solid dosage form of any one of Embodiments 15-20, wherein the at least one pH dependent polymer is hydroxypropylmethyl cellulose acetate succinate (HPMCAS) or alginic acid.

Embodiment 22. The extended release solid dosage form of any one of Embodiments 1-21, wherein the solid dosage form has a total weight less than 400 mg.

Embodiment 23. The extended release solid dosage form of Embodiment 22, wherein the solid dosage form has a total weight between about 100 mg and about 300 mg.

Embodiment 24. In other embodiments, provided is a stable solid dosage form comprising upadacitinib or a pharmaceutically acceptable salt thereof, at least one pH dependent polymer, and at least one release control material;
wherein no more than 0.2% w/w of a upadacitinib degradation product is present in the solid dosage form at an initial timepoint and no more than 0.5% w/w of the upadacitinib degradation product is present in the solid dosage form at a post-storage timepoint;
wherein the initial and post-storage timepoints are separated by at least 3 months, at least 6 months, at least 9 months, at least 12 months, at least 18 months, at least 24 months, at least 30 months, or at least 36 months during which the composition is at 25° C.±2° C. and 60%±5% relative humidity; and
wherein the upadacitinib degradation product is (3S,4R)-3-ethyl-4-(3-(hydroxymethyl)-3H-imidazo[1,2-a]pyrrolo[2,3-e]pyrazin-8-yl)-N-(2,2,2-trifluoroethyl) pyrrolidine-1-carboxamide.

Embodiment 25. The stable solid dosage form of Embodiment 24, wherein the at least one pH dependent polymer is an enteric polymer or an anionic polysaccharide.

Embodiment 26. The stable solid dosage form of Embodiment 25, wherein the at least one pH dependent polymer is hydroxypropylmethyl cellulose acetate succinate (HPMCAS) or alginic acid.

Embodiment 27. The stable solid dosage form of any one of Embodiments 24-26, wherein from about 68% to about 100% of upadacitinib is released from the solid dosage form in about 6 hours using USP I method at a rotation speed of 150 rpm in 900 ml of pH 1.1, 0.1N HCl at 37° C. and/or from about 75% to about 100% of upadacitinib is released from the solid dosage form in about 8 hours using USP I method at a rotation speed of 150 rpm in 900 ml of pH 1.1, 0.1N HCl at 37° C.

Embodiment 28. The stable solid dosage form of Embodiment 27, wherein from about 56% to about 94% of upadacitinib is released from the solid dosage form in about 4 hours using USP I method at a rotation speed of 150 rpm in 900 ml of pH 1.1, 0.1N HCl at 37° C.

Embodiment 29. The stable solid dosage form of any one of Embodiments 24-26, wherein from about 51% to about 85% of upadacitinib is released from the solid dosage form in about 6 hours using USP I method at a rotation speed of 150 rpm in 900 ml of pH 6.8, 0.025 M sodium phosphate buffer containing 2.75% sodium chloride at 37° C. and/or from about 59% to about 99% of upadacitinib is released from the solid dosage form in about 8 hours using USP I method at a rotation speed of 150 rpm in 900 ml of pH 6.8, 0.025 M sodium phosphate buffer containing 2.75% sodium chloride at 37° C.

Embodiment 30. The stable solid dosage form of Embodiment 29, wherein from about 41% to about 68% of upadacitinib is released from the solid dosage form in about 4 hours using USP I method at a rotation speed of 150 rpm in 900 ml pH 6.8, 0.025 M sodium phosphate buffer containing 2.75% sodium chloride at 37° C.

Embodiment 31. The stable solid dosage form of any one of Embodiments 24-26, wherein from about 60% to about 100% of upadacitinib is released from the solid dosage form in about 6 hours using USP I method at a rotation speed of 150 rpm in 900 ml of pH 6.8, 0.025 M sodium phosphate buffer at 37° C. and/or from about 67% to about 100% of upadacitinib is released from the solid dosage form in about 8 hours using USP I method at a rotation speed of 150 rpm in 900 ml of pH 6.8, 0.025 M sodium phosphate buffer at 37° C.

Embodiment 32. The stable solid dosage form of Embodiment 31, wherein from about 49% to about 81% of upadacitinib is released from the solid dosage form in about 4 hours using USP I method at a rotation speed of 150 rpm in 900 ml pH 6.8, 0.025 M sodium phosphate buffer at 37° C.

Embodiment 33. The stable solid dosage form of any one of Embodiments 24-26, wherein from about 58% to about 96% of upadacitinib is released from the solid dosage form in about 6 hours using USP I method at a rotation speed of 150 rpm in 900 ml of pH 6.8, 0.050 M sodium phosphate buffer at 37° C. and/or from about 65% to about 100% of upadacitinib is released from the solid dosage form in about 8 hours using USP I method at a rotation speed of 150 rpm in 900 ml of pH 6.8, 0.050 M sodium phosphate buffer at 37° C.

Embodiment 34. The stable solid dosage form of Embodiment 33, wherein from about 47% to about 79% of upadacitinib is released from the solid dosage form in about 4 hours using USP I method at a rotation speed of 150 rpm in 900 ml pH 6.8, 0.050 M sodium phosphate buffer at 37° C.

Embodiment 35. An extended release solid dosage form comprising upadacitinib, or a pharmaceutically acceptable salt thereof, at least one pH dependent polymer and at least one release control material; and further comprisnig a basic pH modifier.

Embodiment 36. The extended release solid dosage form of Embodiment 35, wherein the basic pH modifier is sodium carbonate, meglumine, or tribasic sodium phosphate dodecahydrate.

Embodiment 37. The extended release solid dosage form of any one of Embodiments 35-36, wherein the basic pH modifier is present in the solid dosage form in an amount from about 5% to about 25% by weight (w/w) of the solid dosage form.

Embodiment 38. The extended release solid dosage form of any one of Embodiments 35-37, wherein the at least one pH dependent polymer and the at least one release control material comprise a matrix system containing upadacitinib or a pharmaceutically acceptable salt thereof.

Embodiment 39. The extended release solid dosage form of any one of Embodiments 35-38, wherein the dosage form does not comprise an enteric film coat.

Embodiment 40. The extended release solid dosage form of any one of Embodiments 35-39, wherein the at least one pH dependent polymer is an enteric polymer or an anionic polysaccharide.

Embodiment 41. The extended release solid dosage form of any one of Embodiments 35-40, wherein the at least one pH dependent polymer is hydroxypropylmethyl cellulose acetate succinate (HPMCAS) or alginic acid.

Embodiment 42. The extended release solid dosage form of any one of Embodiments 35-41, wherein the at least one pH dependent polymer is present in an amount from about 20% to about 40% w/w and the at least one release control material is present in an amount from about 30% to about 60% w/w of the solid dosage form.

Embodiment 43. An extended release solid dosage form comprising upadacitinib, or a pharmaceutically acceptable salt thereof, at least one release rate modifier and at least one release control material, wherein (a) the at least one release rate modifier comprises an ion exchange resin, or (b) the at least one release rate modifier comprises a basic pH modifier and the extended release solid dosage form further comprises an anionic polymer.

Embodiment 44. The extended release solid dosage form of Embodiment 43, wherein the at least one release rate modifier is an ion exchange resin.

Embodiment 45. The extended release solid dosage form of Embodiment 44, wherein the ion exchange resin comprises a sulfonated copolymer comprising styrene and divinylbenzene.

Embodiment 46. The extended release solid dosage form of Embodiment 44, wherein upadacitinib, or a pharmaceutically acceptable salt thereof, and the ion exchange resin form an upadacitinib-ion exchange resin complex, said upadacitinib-ion exchange resin complex comprising upadacitinib, or a pharmaceutically acceptable salt thereof, bound to the ion exchange resin.

Embodiment 47. The extended release solid dosage form of Embodiment 43, wherein the at least one release rate modifier is a basic pH modifier and the extended release solid dosage form further comprises an anionic polymer.

Embodiment 48. The extended release solid dosage form of Embodiment 47, wherein the basic pH modifier is present in an amount from about 5% to about 20% w/w.

Embodiment 49. The extended release solid dosage form of any one of Embodiments 47-48, wherein the basic pH modifier is sodium carbonate, meglumine, or tribasic sodium phosphate dodecahydrate.

Embodiment 50. An extended release solid dosage form comprising: a core comprising upadacitinib, or a pharmaceutically acceptable salt thereof, at least one release rate modifier and at least one release control material; and a barrier layer partially covering the core.

Embodiment 51. The extended release solid dosage form of Embodiment 50, wherein the at least one release rate modifier is an acidic pH modifier.

Embodiment 52. The extended release solid dosage form of any one of Embodiments 50-51, wherein barrier layer comprises a pH-dependent polymer.

Embodiment 53. The extended release solid dosage form of Embodiment 52, wherein the pH-dependent polymer is hydroxypropylmethyl cellulose acetate succinate (HPMCAS).

Embodiment 54. The extended release solid dosage form of any one of Embodiments 35-53, wherein the dosage form comprises less than 10% by weight of a hygroscopic acidic pH modifier.

Embodiment 55. The extended release solid dosage form of any one of Embodiments 35-53, wherein the dosage form is substantially free of a hygroscopic acidic pH modifier.

Embodiment 56. The extended release solid dosage form of any one of Embodiments 54-55, wherein the hygroscopic acidic pH modifier is an organic acid.

Embodiment 57. The extended release solid dosage form of any one of Embodiments 54-55, wherein the hygroscopic organic acid is selected from the group consisting of tartaric acid, citric acid, and maleic acid.

Embodiment 58. The extended release solid dosage form of any one of Embodiments 35-57, wherein the at least one release control material is a release control polymer.

Embodiment 59. The extended release solid dosage form of any one of Embodiments 35-58, wherein the at least one release control polymer is selected from the group consisting of hydroxypropylmethyl cellulose (HPMC), hydroxypropyl cellulose, hydroxyethyl cellulose, a copolymer of acrylic acid crosslinked with a polyalkenyl polyether (Carbopol), non-ionic homopolymer of ethylene oxide (Polyox), a water soluble natural gum of a polysaccharide, crosslinked starch, polyvinyl acetate, polyvinylpyrrolidone, and combinations thereof.

Embodiment 60. The extended release solid dosage form of any one of Embodiments 35-59, wherein the solid dosage form further comprises one or more additional excipients selected from the group consisting of a filler, a binder, a glidant, a lubricant, a film coat, and combinations thereof; wherein the one or more additional excipients are present in an amount less than 50% w/w of the solid dosage form.

Embodiment 61. An extended release solid dosage form comprising a core comprising upadacitinib, or a pharmaceutically acceptable salt thereof; a semi-permeable membrane covering the core, wherin the semi-permeable membrane is permeable to aqueous fluid and substantially impermeable to upadacitinib; and at least one drug delivery orifice that provides a passageway such that upadacitinib, or the pharmaceutically acceptable salt thereof, can be released from the core into an environment external to the extended release solid dosage form.

Embodiment 62. The extended release solid dosage form of Embodiment 61, wherein the core further comprises an osmogent.

Embodiment 63. The extended release solid dosage form of Embodiment 62, wherein the osmogent comprises a water soluble salt of an inorganic acid, an osmotic polymer, a carbohydrate, or a combinations thereof.

Embodiment 64. The extended release solid dosage form of any one of Embodiments 61-63, wherein the core is single-layered or multi-layered.

Embodiment 65. The extended release solid dosage form of any one of Embodiments 61-63, whereinthe core comprises a push layer.

Embodiment 66. The extended release solid dosage form of Embodiment 65, wherein the push layer comprises an osmotic polymer, preferably, hydroxyethylcellulose (HEC).

Embodiment 67. The extended release solid dosage form of any one of Embodiments 61-66, wherein the semi-permeable membrane comprises cellulose acetate (CA).

### EXEMPLIFICATION

The solid dosage forms described herein will be better understood by reference to the following examples, which are included as an illustration of and not a limitation upon the scope of the present disclosure.

### Example 1: RINVOQ Formulations Comprising an Acidic pH Modifier and Release Control Polymer

The formulations for marketed (or to-be-marketed) RINVOQ extended release tablets are shown in Table 1. Upadacitinib tablets comprising 0%, 10%, 20%, and 30% tartaric acid (TA) are provided in Table 2.

TABLE 1
RINVOQ extended release coated tablets

| | | Amount (mg)/Tablet | | |
|---|---|---|---|---|
| Component | Function | 7.5 mg | 15 mg | 30 mg |

Intragranular

| | | | | |
|---|---|---|---|---|
| Upadacitinib Hemihydrate | Drug | 7.7* | 15.4* | 30.7* |
| | substance | | | |
| Hydroxypropylmethyl | Release | 2.5 | 4.9 | 9.8 |
| cellulose (HPMC K4M, | control | | | |
| Hypromellose 2208) | polymer | | | |
| Microcrystalline cellulose | Filler | 20.6 | 41.2 | 82.3 |
| (MCC, Avicel PH102) | | | | |

Extragranular Excipients

| | | | | |
|---|---|---|---|---|
| Microcrystalline cellulose | Filler | 149.5 | 121.3 | 64.8 |
| (MCC, Avicel PH101) | | | | |
| Mannitol | Filler | 100.6 | 100.6 | 100.6 |
| Hydroxypropylmethyl | Release | 93.5 | 91.1 | 86.2 |
| cellulose (HPMC K4M, | control | | | |
| Hypromellose 2208) | polymer | | | |
| Tartaric acid (powdered) | Hygroscopic | 96.0 | 96.0 | 96.0 |
| | Acidic pH | | | |
| | modifier | | | |
| Silica, Colloidal | Glidant | 2.4 | 2.4 | 2.4 |
| anhydrous/Colloidal | | | | |
| silicon dioxide | | | | |
| Magnesium stearate | Lubricant | 7.2 | 7.2 | 7.2 |
| Total (Uncoated Tablet)** | | 480.0 | 480.0 | 480.0 |

Film coating

| | | | |
|---|---|---|---|
| Opadry II PVA*** | 14.4 | 14.4 | 14.4 |
| Total (Coated Tablet) | 494.4 | 494.4 | 494.4 |

TABLE 2
Upadacitinib (coated or uncoated) tablets

| | | Amount per tablet (mg) | | | |
|---|---|---|---|---|---|
| | | 0% | 10% | 20% | 30% |
| Component | Function | TA | TA | TA | TA |

Intragrannular

| | | | | | |
|---|---|---|---|---|---|
| Upadacitinib | Drug | 30.7 | 30.7 | 30.7 | 30.7 |
| Hemihydrate | substance | | | | |
| Hydroxypropylmethyl | Release | 9.54 | 9.54 | 9.54 | 9.54 |
| cellulose (HPMC | control | | | | |
| K4M, Hypromellose | polymer | | | | |
| 2208) | | | | | |
| Microcrystalline | Filler | 79.9 | 79.9 | 79.9 | 79.9 |
| cellulose (MCC, | | | | | |
| Avicel PH101) | | | | | |

Extragranular Excipients

| | | | | | |
|---|---|---|---|---|---|
| Microcrystalline | Filler | 67.2 | 67.2 | 67.2 | 67.2 |
| cellulose (MCC, | | | | | |
| Avicel PH102) | | | | | |
| Mannitol | Filler | 196.6 | 148.6 | 100.6 | 52.6 |
| Hydroxypropylmethyl | Release | 86.5 | 86.5 | 86.5 | 86.5 |
| cellulose (HPMC | control | | | | |
| K4M, Hypromellose | polymer | | | | |
| 2208) | | | | | |
| Tartaric acid | Hygro- | 0.0 | 48.0 | 96.0 | 144.0 |
| (powdered) | scopic | | | | |
| | Acidic pH | | | | |
| | modifier | | | | |
| Silica, Colloidal | Glidant | 2.4 | 2.4 | 2.4 | 2.4 |
| anhydrous/Colloidal | | | | | |
| silicon dioxide | | | | | |
| Magnesium stearate | Lubricant | 7.2 | 7.2 | 7.2 | 7.2 |
| Total (Uncoated | | 480.0 | 480.0 | 480.0 | 480.0 |
| Tablet)** | | | | | |

Film coating

| | | | | | |
|---|---|---|---|---|---|
| Opadry II PVA** | Film coat | 14.4 | 14.4 | 14.4 | 14.4 |
| Total (Coated Tablet) | | 494.4 | 494.4 | 494.4 | 494.4 |

For Tables 1 and 2: *Upadacitinib is provided as Freebase Hydrate Form C, which is a hemihydrate, in an amount to deliver 7.5 mg, 15 mg, or 30 mg of upadacitinib freebase equivalent
**Any difference from total due to rounding.
***Film coating ingredients: polyvinyl alcohol (PVA), macrogol 3350/Polyethylene glycol 3350, talc, titanium dioxide, and iron oxide for color (iron oxide yellow + black iron oxide = green color (7.5 mg); black iron ioxide + iron oxide red = purple color (15 mg); iron oxide red = red color (30 mg); iron oxide yellow = yellow color (all clinical and 45 mg)).

There are several disadvantages of the RINVOQ tablets of Table 1.

FIGS. 1A and 1B depicts mottling of uncoated upadacitinib-containing tablets containing 0%, 10%. 20%, and 30% tartaric acid the 7.5 mg RINVOQ coated tablet. See Tables 1 and 2. In FIG. 1A, increasing mottling/deliquescence is observed in uncoated tablets containing increasing amounts of tartaric acid (TA), i.e., at 0%, 10%, 20%, and 30% TA with a moisture content of 4.2%, when stored at 30° C./53% relative humidity (RH) for 2 months. In FIG. 1B, a visual comparison of “unstressed” 7.5 mg RINVOQ coated tablets of Table 1 versus “stressed” 7.5 mg RINVOQ coated tablets (subjected 40° C./53% RH for >2 months) shows differences in dissolution in pH 6.8 50 mM phosphate buffer. Without being bound by any particular theory, it is believed that the mottling and differences in dissolution are caused by two main respective factors: (a) solubilization of the hydroscopic organic acid in water leading to deliquescence and leaching of tartaric acid from the film coat, such as shown in FIG. 1A, and (b) cross-linking of the solubilized tartaric acid with the film coat containing polyvinylalcohol (PVA), such as shown by the changes in dissolution in FIG. 1B. Stressed RINVOQ tablets exhibited slow dissolution because at least part of the film coat does not completely dissolve due to cross-linking.

Another disadvantage of the RINVOQ Tablets of Table 1 is increased impurity level of the non-genotoxic upadacitinib hydroxymethyl impurity (UHM impurity) over time. As depicted in FIG. 2, the UHM impurity is formed via a reaction with water, upadacitinib, tartaric acid, and trace formaldehyde that is present in excipients, such as polyethylene glycol, HPMC, and PVA. The UHM impurity can be characterized using standard techniques, such as by NMR, HPLC, and/or KF titration.

The UHM impurity has been observed at levels up to 0.19% (practical quantitation limit of the test was 0.10%) in RINVOQ film coated tablets of Table 1 that have been stored for 6 months at 40° C./75% RH. At 12 months, 30° C./75% RH, the UHM impurity has been observed at levels up to 0.07% (practical quantitation limit of the test was 0.03%). Stability data indicated growth of the UHM impurity over time, particularly for tablets containing 7.5 mg of upadacitinib freebase equivalent and/or tablets stored in a blister package.

Table 3 summarizes stability data from a solid dosage form described herein (an AS2 formulation blend; see Example 3) and historical stability data from RINVOQ extended release tablets and formulation blends comprising the excipients of the RINVOQ tablets. A “formulation blend” refers to a loose powder blend prior to compression into a tablet.

TABLE 3
Percentage of UHM Impurity under Stressed Conditions

| Study | | Storage | | % UHM |
|---|---|---|---|---|
| (Conditions) | Sample | time | | Impurity |
| Study #1 | RINVOQ tablet of | Initial | | 0.02 |
| (40° C./ | Table 1, uncoated | 1 | mo. | 0.04 |
| 53% RH) | 7.5 mg | 2 | mo. | 0.05 |
| | | 3 | mo. | 0.05 |
| | RINVOQ tablet of | Initial | | 0.02 |
| | Table 1, film coated | 1 | mo. | 0.29 |
| | 7.5 mg | 2 | mo. | 0.25 |
| | | 3 | mo. | 0.25 |
| Study #2 | Formulation blend of | 5 | days | 0.15 |
| (80° C./ | RINVOQ components | | | |
| 75% RH) | of Table 1 15 mg | | | |
| Study #3 | Formulation blend | 4 | weeks | Not |
| (60° C./ | for AS2 tablet of | | | detected |
| 50% RH) | Table 6 15 mg | | | |

### Example 2. Smaller Tablets With and Without TA and Comparison to RINVOQ

The present Example sought to address another disadvantage of the RINVOQ tablets of Table 1, i.e., the relatively large size of the tablets (˜500 mg).

Formulation A1 (without tartaric acid): 3.84 g of Upadacitinib, 12.5 g of HPMC K4M, 16.4 g of Avicel PH102, 1.50 g of hydroxypropyl cellulose, 0.25 g of colloidal silicon dioxide, and 15.00 g of mannitol were sieved through 30 mesh screen, added to 250 ml bottle and mixed for approximately 5 minutes on Turbula blender. The powder blend was subsequently mixed with 0.50 g of sodium stearyl fumarate, followed by mixing for approximately 2 minutes on Turbula blender and compression into 200 mg ovaloid shape tablets on a Carver press.

Formulation T1 (with tartaric acid): 7.68 g of Upadacitinib, 12.5 g of HPMC K4M, 2.57 g of Avicel PH102, 1.50 g of hydroxypropyl cellulose, 0.25 g of colloidal silicon dioxide, 15.00 g of mannitol, and 10.00 g of tartaric acid were sieved through 30 mesh screen, added to 250 ml bottle and mixed for approximately 5 minutes on Turbula blender. The powder blend was subsequently mixed with 0.50 g of sodium stearyl fumarate, followed by mixing for approximately 2 minutes on Turbula blender and compression into 200 mg ovaloid shape tablets on a Carver press. The formulations for smaller sized tablets are provided in Table 4.

TABLE 4
Smaller sized tablets with and without Tartaric acid

| | | A1 | | T1 | |
|---|---|---|---|---|---|
| | | (15 mg w/o TA) | | (30 mg w/TA) | |
| Component | Function | mg | % | mg | % |
| Upadacitinib* | Drug | 15.4 | 7.7 | 30.7 | 15.4 |
| | substance | | | | |
| Hydroxypropylmethyl | Release | 50.0 | 25.0 | 50.0 | 25.0 |
| cellulose (HPMC | control | | | | |
| K4M, Hypromellose | polymer | | | | |
| 2208) | | | | | |
| Tartaric acid | Hygroscopic | — | — | 40.0 | 20.0 |
| | Acidic pH | | | | |
| | modifier | | | | |
| Hydroxypropyl | Binder | 6.0 | 3.0 | 6.0 | 3.0 |
| cellulose (HPC) | | | | | |
| Microcrystalline | Filler | 65.6 | 32.8 | 10.3 | 5.2 |
| cellulose (MCC, | | | | | |
| Avicel PH102) | | | | | |
| Mannitol | | 60.0 | 30.0 | 60.0 | 30.0 |
| Colloidal silicon | glidant | 1.0 | 0.5 | 1.0 | 0.5 |
| dioxide | | | | | |
| Sodium stearyl | lubricant | 2.0 | 1.0 | 2.0 | 1.0 |
| fumarate | | | | | |
| Total (Uncoated | | 200.0 | 100.0 | 200.0 | 100.0 |
| Tablet)** | | | | | |

*Upadacitinib was provided as Freebase Hydrate Form C, which is a hemihydrate, in an amount to deliver 15 mg or 30 mg of upadacitinib freebase equivalent.
**Any difference from total due to rounding.

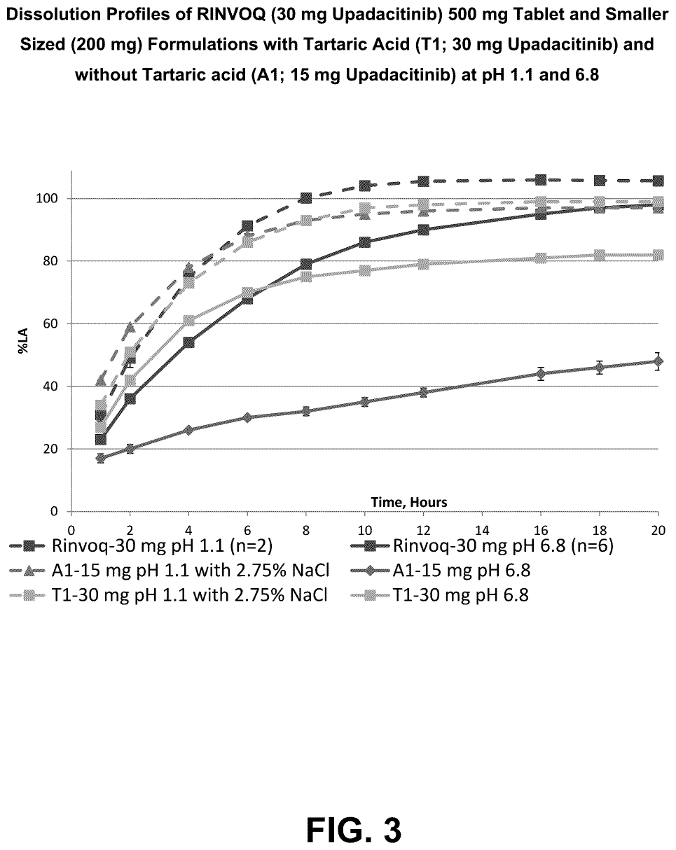

In vitro dissolution rates of formulations A1 (without TA) and T1 (with TA) were determined using USP I method at a rotation speed of 150 rpm in 900 ml at 37° C. of (1) pH 6.8, 0.025 M sodium phosphate buffer containing 2.75% sodium chloride, and (2) pH 1.1, 0.1N HCl, and compared to the RINVOQ 30 mg formulation of Table 1. Test results are provided in Tables 5A and 5B and FIG. 3.

TABLE 5A
pH-Dissolution Profiles of FIG. 3

| | Formulation | | | |
|---|---|---|---|---|
| | A1 | | T1 | |
| | (15 mg w/o TA) | | (30 mg w/TA) | |
| | pH | | | |
| | pH 1.1^{~} | pH 6.8^{\hat{ }} | pH 1.1^{~} | pH 6.8^{\hat{ }} |
| Time | Release % | | | |
| 1 | 42 | 17 | 34 | 27 |
| 2 | 59 | 20 | 51 | 42 |
| 4 | 78 | 26 | 73 | 61 |
| 6 | 88 | 30 | 86 | 70 |
| 8 | 93 | 32 | 93 | 75 |
| 10 | 95 | 35 | 97 | 77 |
| 12 | 96 | 38 | 98 | 79 |
| 16 | 97 | 44 | 99 | 81 |
| 18 | 97 | 46 | 99 | 82 |
| 20 | 97 | 48 | 99 | 82 |

^{~}pH 1.1, 0.1N HCl with 2.75% NaCl at a rotation speed of 150 rpm;
^{\hat{ }}pH 6.8, 0.025M sodium phosphate buffer containing 2.75% sodium chloride at a rotation speed of 150 rpm

TABLE 5B
pH-Dissolution Profiles of FIG. 3

| | Formulation | | | |
|---|---|---|---|---|
| | RINVOQ 30 mg | | | |
| | pH | | | |
| Time | pH 1.1^{~} | pH 1.1^{~} | pH 6.8^{\hat{ }} | pH 6.8^{\hat{ }} |
| (hr) | Release % | | | |
| 1 | 31 | 23-39 | 23 | 17-29 |
| 2 | 49 | 37-61 | 36 | 27-45 |
| 4 | 75 | 56-94 | 54 | 41-68 |
| 6 | 91 | 68-≥100 | 68 | 51-85 |
| 8 | 100 | 75-≥100 | 79 | 59-99 |
| 10 | 104 | 75-≥100 | 86 | 65-≥100 |
| 12 | 105 | 75-≥100 | 90 | 68-≥100 |
| 16 | 106 | 75-≥100 | 95 | 71-≥100 |
| 18 | 106 | 75-≥100 | 97 | 73-≥100 |
| 20 | 106 | 75-≥100 | 98 | 74-≥100 |

^{~}pH 1.1, 0.1N HCl with 2.75% NaCl at a rotation speed of 150 rpm;
^{\hat{ }}pH 6.8, 0.025M sodium phosphate buffer containing 2.75% sodium chloride at a rotation speed of 150 rpm

The data demonstrates the difficulty in designing smaller tablets of upadacitinib having a similar extended release profile to the RINVOQ formulation, but with less (or no) tartaric acid. For example, the smaller sized tablet formulation T1 (200 mg), containing 20% tartaric acid, exhibited a comparable release profile to RINVOQ (30 mg) at a pH of 1.1 and pH 6.8, although at a pH 6.8, substantially complete release was not observed. The smaller sized tablet formulation A1 (200 mg), albeit at the 15 mg dose, with no tartaric acid present, demonstrated a comparable release profile to RINVOQ (30 mg) at a pH 1.1, but demonstrated a different release profile at a pH 6.8. Smaller sized tablets have an increased surface-to-mass ratio which may be one of the factors impairing release rate at different pHs. However, in general, it was found tartaric acid containing formulations were less effective in smaller sized tablets; see, e.g., Example 4 which explores use of tartaric acid in a pH-dependent polymer/release control material formulation. Thus, alternate approaches were required to achieve similar drug release to that of marketed RINVOQ when the tablet size is reduced.

### Example 3: Enteric Polymer as a pH-Dependent Polymer

In order to explore alternate approaches for achieving a smaller tablet size and improved physical and chemical stability of the RINVOQ tablet, smaller sized upadacitinib extended release formulations were prepared comprising an enteric polymer and release control material using direct compression process.

Formulation AS1: 3.69 g of upadacitinib, 12.00 g of HPMC K4M, 9.00 g of HPMCAS, 5.01 g of Avicel PH102 were sieved through 35 mesh screen, added to 125 mL bottle and mixed for approximately 5 minutes on Turbula blender. The powder blend was subsequently mixed with magnesium stearate, followed by compression into 250 mg ovaloid shape tablets on a Carver press.

Formulation AS2: 3.84 g of Upadacitinib, 20.0 g of HPMC K4M, 8.91 g of Avicel PH102, 1.50 g of hydroxypropyl cellulose, 0.25 g of colloidal silicon dioxide and 15.00 g of HPMCAS were sieved through 30 mesh screen, added to 250 ml bottle and mixed for approximately 5 minutes on Turbula blender. The powder blend was subsequently mixed with 0.50 g of sodium stearyl fumarate, followed by mixing for approximately 2 minutes on Turbula blender and compression into 200 mg ovaloid shape tablets on a Carver press.

Formulation AS3: 10.24 g of Upadacitinib, 22.5 g of HPMC K4M, 0.013 g of Avicel PH102, 1.5 g of hydroxypropyl cellulose, 0.25 g of colloidal silicon dioxide and 15.0 g of HPMCAS were sieved through 30 mesh screen, added to 250 ml bottle and mixed for approximately 5 minutes on Turbula blender. The powder blend was subsequently mixed with 0.50 g of sodium stearyl fumarate, followed by mixing for approximately 2 minutes on Turbula blender and compression into 150 mg ovaloid shape tablets on a Carver press.

Formulation AS4: 7.68 g of Upadacitinib, 12.5 g of HPMC K4M, 12.57 g of Avicel PH102, 5 g of mannitol, 1.5 g of hydroxypropyl cellulose, 0.25 g of colloidal silicon dioxide and 10 g of HPMCAS were sieved through 30 mesh screen, added to 250 ml bottle and mixed for approximately 5 minutes on Turbula blender. The powder blend was subsequently mixed with 0.50 g of sodium stearyl fumarate, followed by mixing for approximately 2 minutes on Turbula blender and compression into 200 mg ovaloid shape tablets on a Carver press.

Formulation AS5: 7.68 g of Upadacitinib, 10 g of HPMC K4M, 7.57 g of Avicel PH102, 12.5 g of mannitol, 1.5 g of hydroxypropyl cellulose, 0.25 g of colloidal silicon dioxide and 10 g of HPMCAS were sieved through 30 mesh screen, added to 250 ml bottle and mixed for approximately 5 minutes on Turbula blender. The powder blend was subsequently mixed with 0.50 g of sodium stearyl fumarate, followed by mixing for approximately 2 minutes on Turbula blender and compression into 200 mg ovaloid shape tablets on a Carver press.

Formulation AS6: 1.54 g of Upadacitinib, 12 g of HPMC K4M, 9 g of HPMCAS, 7.16 g of lactose were sieved through 35 mesh screen, added to 125 mL bottle and mixed for approximately 5 minutes on Turbula blender. The powder blend was subsequently mixed with magnesium stearate, followed by compression into 300 mg ovaloid shape tablets on a Carver press.

Formulation AS7: 7.68 g of Upadacitinib, 20.0 g of HPMC K4M, 4.07 g of Avicel PH102, 2.50 g of hydroxypropyl cellulose, 0.25 g of colloidal silicon dioxide and 15.00 g of HPMCAS were sieved through 30 mesh screen, added to 250 ml bottle and mixed for approximately 5 minutes on Turbula blender. The powder blend was subsequently mixed with 0.50 g of magnesium stearate, followed by mixing for approximately 2 minutes on Turbula blender and compression into 200 mg ovaloid shape tablets on a Carver press.

The formulations of AS1-AS7 are shown in Table 6A and 6B.

TABLE 6A
HPMC-AS Formulations (AS1-AS4)

| | | AS1 | | AS2** | | AS3 | | AS4 | |
|---|---|---|---|---|---|---|---|---|---|
| | | (30 mg) | | (15 mg) | | (30 mg) | | (30 mg) | |
| | | | %^{a} | | %^{a} | | %^{a} | | %^{a} |
| Component | Function | mg | (w/w) | mg | (w/w) | mg | (w/w) | mg | (w/w) |
| Upadacitinib* | drug | 30.7 | 12.3 | 15.4 | 7.7 | 30.7 | 20.5 | 30.7 | 15.4 |
| | substance | | | | | | | | |
| Hydroxypropylmethyl | release | 100.0 | 40.0 | 80.0 | 40.0 | 67.5 | 45.0 | 50.0 | 25.0 |
| cellulose | control | | | | | | | | |
| (HPMC K4M, | material | | | | | | | | |
| Hypromellose 2208) | | | | | | | | | |
| HPMC-acetate | pH- | 75.0 | 30.0 | 60.0 | 30.0 | 45.0 | 30.0 | 40.0 | 20.0 |
| succinate (AS) | dependent | | | | | | | | |
| | polymer | | | | | | | | |
| Hydroxypropyl | binder | — | — | 6.0 | 3.0 | 4.5 | 3.0 | 6.0 | 3.0 |
| cellulose (HPC) | | | | | | | | | |
| Microcrystalline | filler | 41.8 | 16.7 | 35.6 | 17.8 | 0.04 | 0.03 | 50.3 | 25.1 |
| cellulose | | | | | | | | | |
| (MCC, Avicel PH102) | | | | | | | | | |
| Lactose | | — | — | — | — | — | — | — | — |
| Mannitol | | — | — | — | — | — | — | 20.0 | 10.0 |
| Colloidal silicon | glidant | — | — | 1.0 | 0.5 | 0.80 | 0.5 | 1.0 | 0.5 |
| dioxide | | | | | | | | | |
| Sodium stearyl | lubricant | — | — | 2.0 | 1.0 | 1.5 | 1.0 | 2.0 | 1.0 |
| fumarate | | | | | | | | | |
| Magnesium stearate | | 2.5 | 1.0.0 | — | — | — | — | — | — |

| | | | | | | | | |
|---|---|---|---|---|---|---|---|---|
| Tablet Weight | 250 | 100.0 | 200.0 | 100.0 | 150.0 | 100.0 | 200.0 | 100.0 |

^{a}Percents given based on the total tablet weight. Total percentage may not be 100% due to rounding.
*Upadacitinib was provided as Freebase Hydrate Form C, which is a hemihydrate, in an amount to deliver 15 mg or 30 mg of upadacitinib freebase equivalent.
**AS2 formulation blend referred to in Table 3 are the formulation components of Table 6A as a powder without direct compression.

TABLE 6B
HPMC-AS Formulations (AS5-AS7)

| | | AS5 | | AS6 | | AS7 | |
|---|---|---|---|---|---|---|---|
| | | (30 mg) | | (15 mg) | | (30 mg) | |
| | | | %^{a} | | %^{a} | | %^{a} |
| Component | Function | mg | (w/w) | mg | (w/w) | mg | (w/w) |
| Upadacitinib* | drug substance | 30.7 | 15.4 | 15.4 | 5.1 | 30.7 | 15.4 |
| Hydroxypropylmethyl | release | 40.0 | 20.0 | 120.0 | 40.0 | 80 | 40.0 |
| cellulose | control | | | | | | |
| (HPMC K4M, | material | | | | | | |
| Hypromellose 2208) | | | | | | | |
| HPMC-acetate | pH-dependent | 40.0 | 20.0 | 90.0 | 30.0 | 60.0 | 30.0 |
| succinate (AS) | polymer | | | | | | |
| Hydroxypropyl | binder | 6.0 | 3.0 | — | — | 10.0 | 5.0 |
| cellulose (HPC) | | | | | | | |
| Microcrystalline | filler | 30.3 | 15.1 | — | — | 16.3 | 8.1 |
| cellulose | | | | | | | |
| (MCC, Avicel PH102) | | | | | | | |
| Lactose | | — | — | 71.6 | 23.9 | — | — |
| Mannitol | | 50.0 | 25.0 | — | — | — | — |
| Colloidal silicon | glidant | 1.0 | 0.5 | — | — | 1.0 | 0.5 |
| dioxide | | | | | | | |
| Sodium stearyl | lubricant | 2.0 | 1.0 | — | — | — | — |
| fumarate | | | | | | | |
| Magnesium stearate | | — | — | 3.0 | 1.0 | 2.0 | 1.0 |

| | | | | | | |
|---|---|---|---|---|---|---|
| Tablet Weight | 200.0 | 100.0 | 300.0 | 100.0 | 200.0 | 100.0 |

^{a}Percents given based on the total tablet weight. Total percentage may not be 100% due to rounding.
*Upadacitinib was provided as Freebase Hydrate Form C, which is a hemihydrate, in an amount to deliver 15 mg or 30 mg of upadacitinib freebase equivalent.

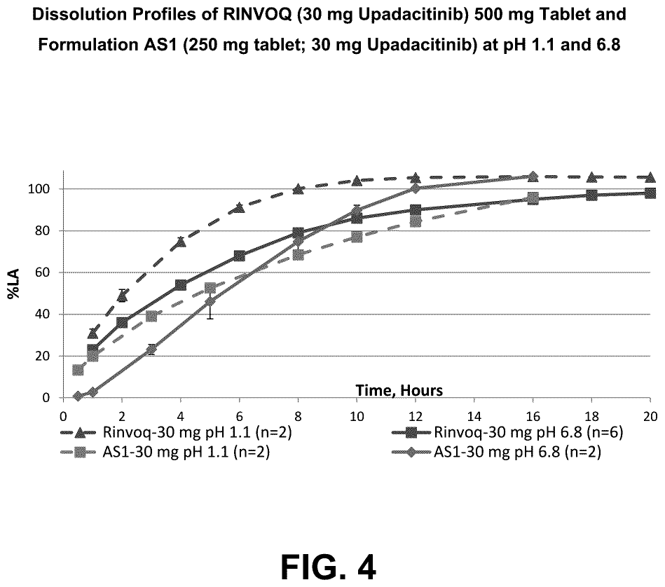

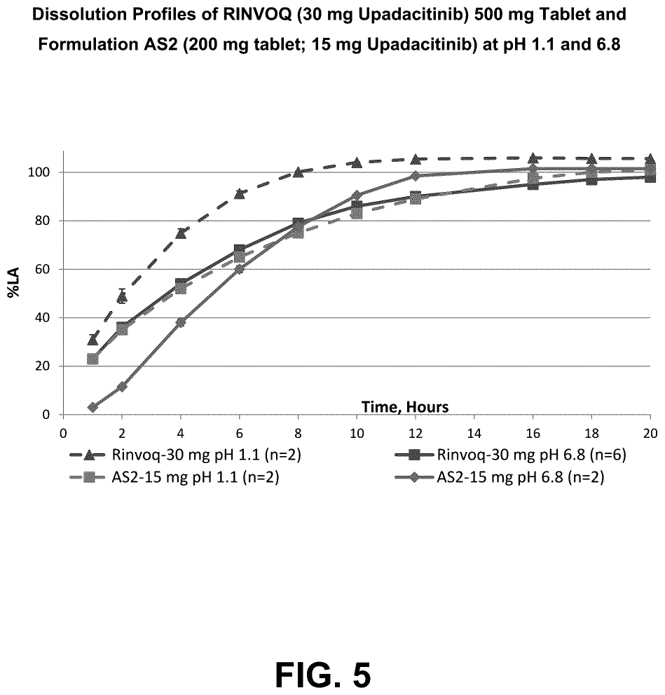

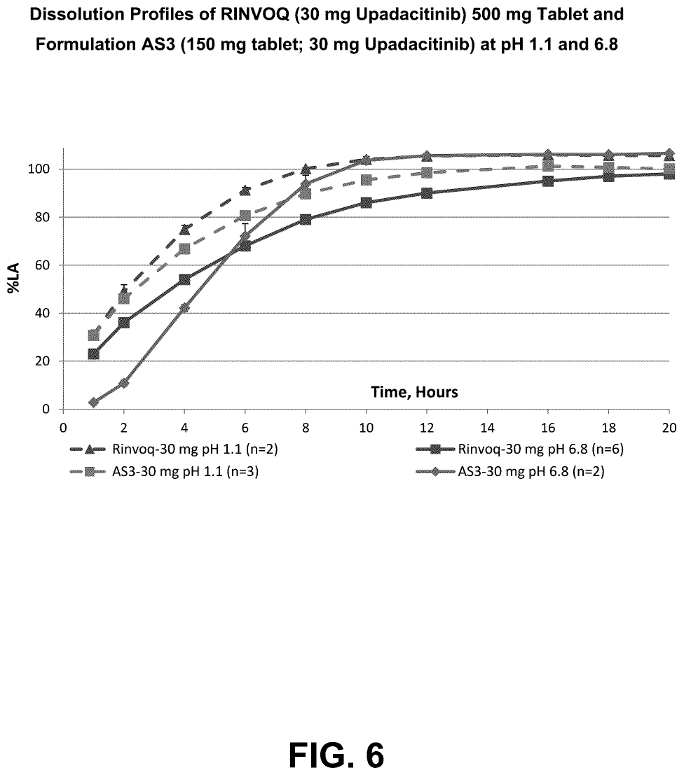

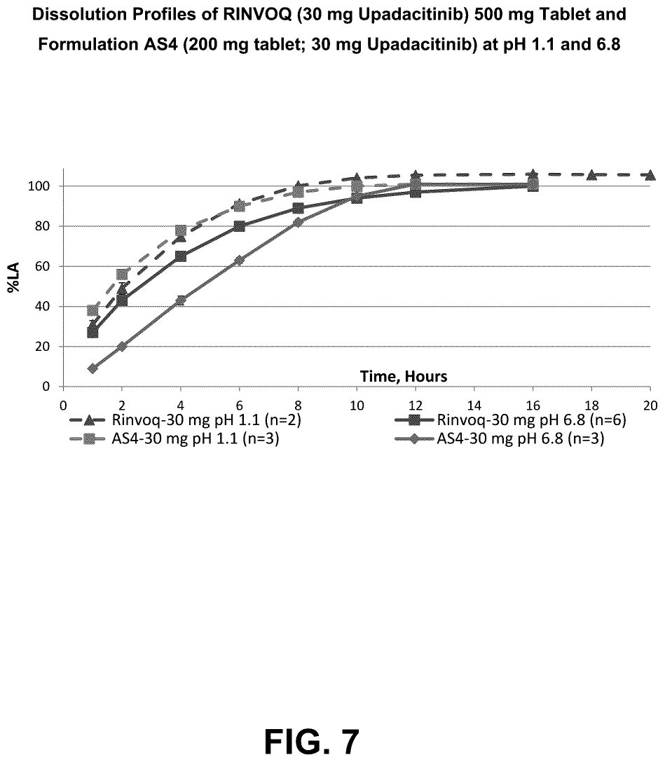

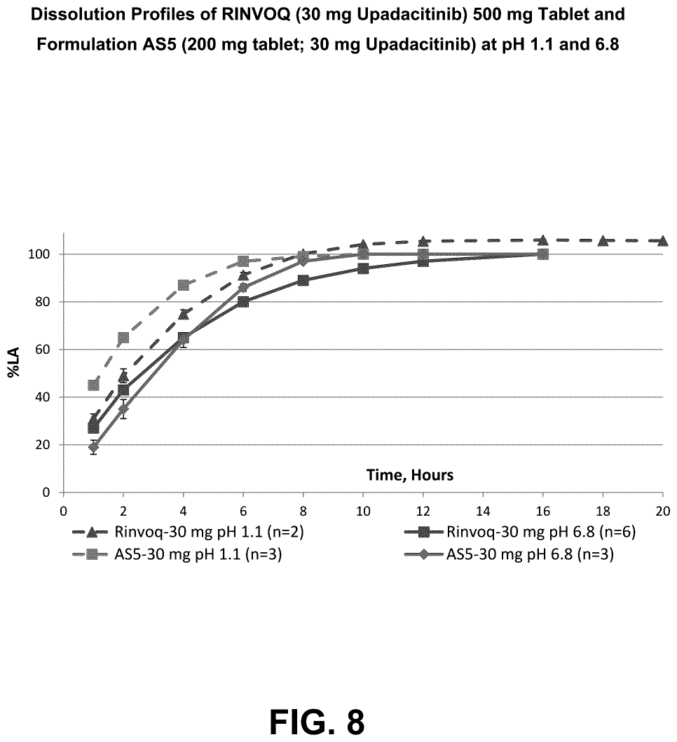

In vitro dissolution rates of formulations AS1, AS2, and AS3 were determined using USP I method at a rotation speed of 150 rpm in 900 ml at 37° C. of (1) pH 6.8, 0.025 M sodium phosphate buffer containing 2.75% sodium chloride, and (2) pH 1.1, 0.1N HCl. In vitro dissolution rates of formulations AS4 and AS5 were determined using USP I method at a rotation speed of 150 rpm in 900 ml at 37° C. of (1) pH 6.8, 0.025 M sodium phosphate buffer, and (2) pH 1.1, 0.1N HCl. RINVOQ (30 mg) formulation of Table 1 dissolution profiles are provided in Table 4. Test results provided in Tables 7A and 7B and FIGS. 4-8 show that comparable extended drug release was obtained for each of the smaller sized tablet formulations AS1-AS5 to the larger sized RINVOQ (30 mg) formulation, with extended release up to 10 to 12 hours and substantially steady release was observed at pH 1.1 and 6.8 for AS1-AS3, and extended release up to 8 to 10 hours and substantially steady release for AS4 and AS5. The rate differences at pH 1.1 and 6.8 for formulations AS1-AS5 versus RINVOQ were small despite of the significant solubility difference of upadacitinib at pH 1.1 and pH 6.8 (i.e., solubility of upadacitinib ranges from 38.4±1.5 mg/mL in 0.1 N HCl medium at 37° C., pH 1.0 to 0.194±0.001 mg/mL in 50 mM sodium phosphate buffer at 37° C., pH 7.02).

TABLE 7A
pH-Dissolution Profiles of AS1-AS5 of FIGS. 4-8

| | Formulation | | | | | | | | | |
|---|---|---|---|---|---|---|---|---|---|---|
| | AS1 | | AS2 | | AS3 | | AS4 | | AS5 | |
| | (30 mg) | | (15 mg) | | (30 mg) | | (30 mg) | | (30 mg) | |
| | pH | | | | | | | | | |
| | pH | pH | pH | pH | pH | pH | pH | pH | pH | pH |
| | 1.1~ | 6.8^{\hat{ }} | 1.1~ | 6.8^{\hat{ }} | 1.1~ | 6.8^{\hat{ }} | 1.1~ | 6.8* | 1.1~ | 6.8* |
| Time (hr) | Release % | | | | | | | | | |
| 0.5 | 13 | 1 | — | — | — | — | — | — | — | — |
| 1 | 20 | 3 | 23 | 3 | 31 | 3 | 38 | 9 | 45 | 19 |
| 2 | — | — | 35 | 12 | 46 | 11 | 56 | 20 | 65 | 35 |
| 3 | 39 | 23 | — | — | — | — | — | — | — | — |
| 4 | — | — | 52 | 38 | 67 | 42 | 78 | 43 | 87 | 64 |
| 5 | 53 | 46 | — | — | — | — | — | — | — | — |
| 6 | — | — | 65 | 60 | 81 | 72 | 90 | 63 | 97 | 86 |
| 8 | 69 | 75 | 75 | 78 | 90 | 94 | 97 | 82 | 99 | 97 |
| 10 | 77 | 90 | 83 | 91 | 96 | 104 | 100 | 95 | 100 | 100 |
| 12 | 84 | 100 | 89 | 99 | 99 | 106 | 101 | 101 | 100 | 100 |
| 16 | 96 | 106 | 98 | 102 | 101 | 106 | 101 | 101 | 100 | 100 |
| 18 | — | — | 100 | 102 | 101 | 106 | — | — | — | — |
| 20 | — | — | 101 | 102 | 100 | 107 | — | — | — | — |

^{\hat{ }}pH 6.8, 0.025M sodium phosphate buffer containing 2.75% sodium chloride at a rotation speed of 150 rpm;
*pH 6.8, 0.025M sodium phosphate buffer at a rotation speed of 150 rpm;
~pH 1.1, 0.1N HCl with 2.75% NaCl at a rotation speed of 150 rpm

TABLE 7B
pH-Dissolution Profiles of RINVOQ (30 mg) of FIGS. 4-8

| | Formulation | | | | | |
|---|---|---|---|---|---|---|
| | RINVOQ (30 mg) | | | | | |
| | pH | | | | | |
| | pH | pH | pH | pH | pH | pH |
| | 1.1^{~} | 1.1^{~} | 6.8^{\hat{ }} | 6.8^{\hat{ }} | 6.8* | 6.8* |
| Time (hr) | Release % | | | | | |
| 1 | 31 | 23-39 | 23 | 17-29 | 27 | 20-34 |
| 2 | 49 | 37-61 | 36 | 27-45 | 43 | 32-54 |
| 4 | 75 | 56-94 | 54 | 41-68 | 65 | 49-81 |
| 6 | 91 | 68-≥100 | 68 | 51-85 | 80 | 60-≥100 |
| 8 | 100 | 75-≥100 | 79 | 59-99 | 89 | 67-≥100 |
| 10 | 104 | 75-≥100 | 86 | 65-≥100 | 94 | 71-≥100 |
| 12 | 105 | 75-≥100 | 90 | 68-≥100 | 97 | 73-≥100 |
| 16 | 106 | 75-≥100 | 95 | 71-≥100 | 100 | 75-≥100 |
| 18 | 106 | 75-≥100 | 97 | 73-≥100 | — | — |
| 20 | 106 | 75-≥100 | 98 | 74-≥100 | — | — |

^{\hat{ }}pH 6.8, 0.025M sodium phosphate buffer containing 2.75% sodium chloride at a rotation speed of 150 rpm;
*pH 6.8, 0.025M sodium phosphate buffer at a rotation speed of 150 rpm;
^{~}pH 1.1, 0.1N HCl with 2.75% NaCl at a rotation speed of 150 rpm

### Example 4: Anionic Polysaccharides as a pH-Dependent Polymer

In order to explore alternate approaches for achieving a smaller tablet size and improved physical and chemical stability of the RINVOQ tablet, smaller sized upadacitinib extended release formulations were also prepared comprising an anionic polysaccharide (alginic acid) and release control material using direct compression process.

Formulation AL1: 4.6 g of upadacitinib, 7.5 g of HPMC K4M, 9 g of alginic acid, 4.5 g of Avicel PH102, 3 g of mannitol, 0.9 g of hydroxypropyl cellulose, and 0.15 g of colloidal silicon dioxide were sieved through 35 mesh screen, added to 125 mL bottle and mixed for approximately 5 minutes on Turbula blender. The powder blend was subsequently mixed with sodium stearyl fumarate, followed by mixing for approximately 2 minutes on Turbula blender and compression into 200 mg ovaloid shape tablets on a Carver press.

Formulation AL2: 2.3 g of upadacitinib, 9.00 g of HPMC K4M, 7.5 g of alginic acid, 6.8 g of Avicel PH102, 3 g of mannitol, 0.9 g of hydroxypropyl cellulose, and 0.15 g of colloidal silicon dioxide were sieved through 35 mesh screen, added to 125 mL bottle and mixed for approximately 5 minutes on Turbula blender. The powder blend was subsequently mixed with sodium stearyl fumarate, followed by mixing for approximately 2 minutes on Turbula blender and compression into 200 mg ovaloid shape tablets on a Carver press.

Formulation AL3: 2.3 g of upadacitinib, 10.5 g of HPMC K4M, 7.5 g of alginic acid, 5.3 g of Avicel PH102, 3 g of mannitol, 0.9 g of hydroxypropyl cellulose, and 0.15 g of colloidal silicon dioxide were sieved through 35 mesh screen, added to 125 mL bottle and mixed for approximately 5 minutes on Turbula blender. The powder blend was subsequently mixed with sodium stearyl fumarate, followed by mixing for approximately 2 minutes on Turbula blender and compression into 200 mg ovaloid shape tablets on a Carver press.

The formulations tablets containing alginic acid (AL) are shown in Table 8.

TABLE 8
Extended release tablets containing Alginic acid (AL)

| | | AL1 | | AL2 | | AL3 | |
|---|---|---|---|---|---|---|---|
| | | (30 mg) | | (15 mg) | | (15 mg) | |
| | | | %^{a} | | %^{a} | | %^{a} |
| Component | Function | mg | (w/w) | mg | (w/w) | mg | (w/w) |
| Upadacitinib* | drug substance | 30.7 | 15.4 | 15.4 | 7.7 | 15.4 | 7.7 |
| Hydroxypropylmethyl | release | 50.0 | 25.0 | 60.0 | 30.0 | 70.0 | 35.0 |
| cellulose | control | | | | | | |
| (HPMC K4M, | polymer | | | | | | |
| Hypromellose 2208) | | | | | | | |
| Alginic acid | pH dependent | 60.0 | 30.0 | 50.0 | 25.0 | 50.0 | 25.0 |
| | polymer | | | | | | |
| Hydroxypropyl | binder | 6.0 | 3.0 | 6.0 | 3.0 | 6.0 | 3.0 |
| cellulose (HPC) | | | | | | | |
| Microcrystalline | filler | 30.3 | 15.1 | 45.6 | 22.8 | 35.6 | 17.8 |
| cellulose | | | | | | | |
| (MCC, Avicel PH102) | | | | | | | |
| Lactose | | — | — | — | — | — | — |
| Mannitol | | 20.0 | 10.0 | 20.0 | 10.0 | 20.0 | 10.0 |
| Colloidal silicon | glidant | 1.0 | 0.5 | 1.0 | 0.5 | 1.0 | 0.5 |
| dioxide | | | | | | | |
| Sodium stearyl | lubricant | 2.0 | 1.0 | 2.0 | 1.0 | 2.0 | 1.0 |
| fumarate | | | | | | | |
| Magnesium stearate | | — | — | — | — | — | — |

| | | | | | | |
|---|---|---|---|---|---|---|
| Tablet Weight | 200.0 | 100.0 | 200.0 | 100.0 | 200.0 | 100.0 |

^{a}Percents given based on the total tablet weight. Total percentage may not be 100% due to rounding.
*Upadacitinib was provided as Freebase Hydrate Form C, which is a hemihydrate, in an amount to deliver 15 mg or 30 mg of upadacitinib freebase equivalent.

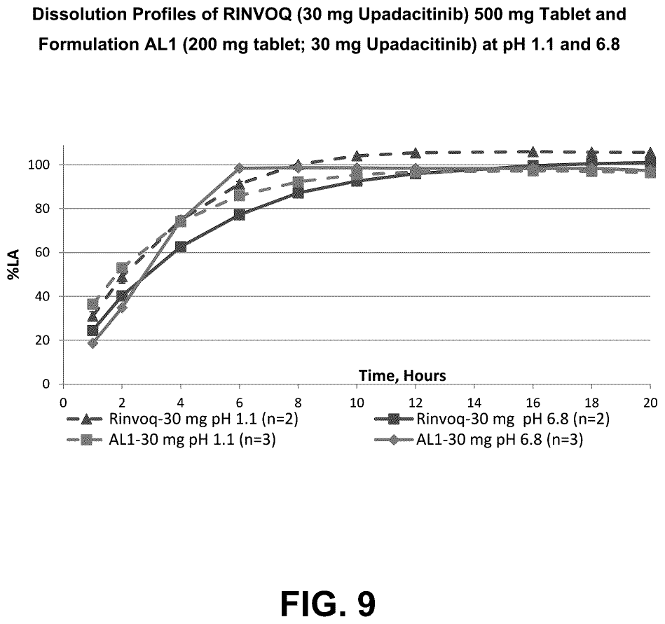

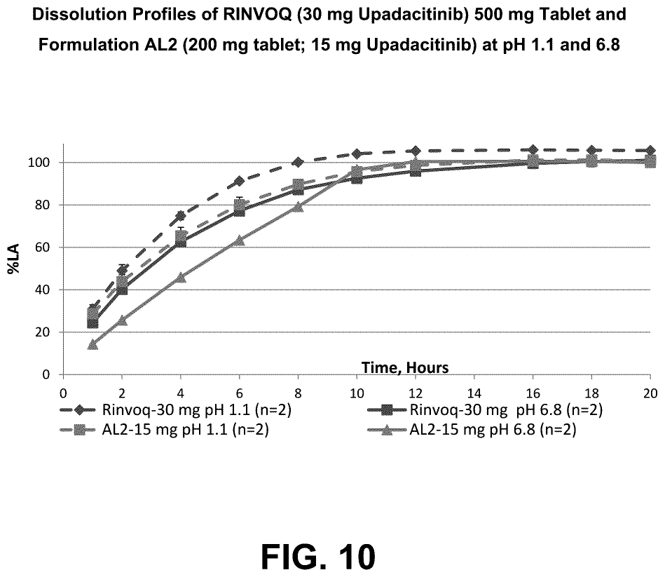

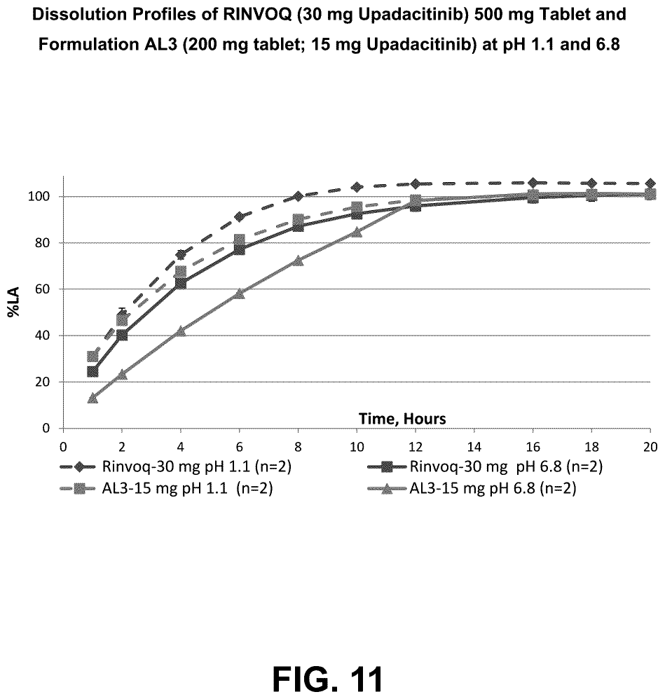

In vitro dissolution rates of formulations AL1, AL2, and AL3 were determined using USP I method at a rotation speed of 150 rpm at 37° C. in 900 ml (1) pH 6.8, 0.050 M sodium phosphate buffer and (2) pH 1.1, 0.1N HCl. Test results provided in Tables 9A and 9B and FIGS. 9-11 show a comparable drug release profile to that of smaller sized tablets AS1-AS5 as well as the larger sized RINVOQ (30 mg) formulation, with extended release up to obtained for up to 8-12 hours and substantially steady release were observed at pH 1.1 and 6.8 As also noted for AS1-AS5, the rate differences at pH 1.1 and 6.8 for formulations AL1-AL3 versus RINVOQ were small despite of the significant solubility difference of upadacitinib at pH 1.1 and pH 6.8 (i.e., solubility of upadacitinib ranges from 38.4±1.5 mg/mL in 0.1 N HCl medium at 37° C., pH 1.0 to 0.194±0.001 mg/mL in 50 mM phosphate buffer at 37° C., pH 7.02).

TABLE 9A
pH-Dissolution Profiles of AL1-AL3 of FIGS. 9-11

| | Formulation | | | | | |
|---|---|---|---|---|---|---|
| | AL1 | | AL2 | | AL3 | |
| | pH | | | | | |
| | pH | pH | pH | pH | pH | pH |
| | 1.1~ | 6.8^{#} | 1.1~ | 6.8^{#} | 1.1~ | 6.8^{#} |
| Time (hr) | Release % | | | | | |
| 1 | 36 | 19 | 29 | 14 | 31 | 13 |
| 2 | 53 | 35 | 44 | 26 | 47 | 23 |
| 4 | 74 | 75 | 65 | 46 | 68 | 42 |
| 6 | 86 | 98 | 80 | 63 | 81 | 58 |
| 8 | 92 | 99 | 90 | 79 | 90 | 72 |
| 10 | 95 | 99 | 96 | 97 | 96 | 85 |
| 12 | 97 | 98 | 99 | 100 | 99 | 98 |
| 16 | 97 | 98 | 101 | 101 | 101 | 101 |
| 18 | 97 | 98 | 101 | 100 | 101 | 101 |
| 20 | 96 | 97 | 101 | 100 | 101 | 101 |

^{#}pH 6.8, 0.050M sodium phosphate buffer at a rotation speed of 150 rpm;
~pH 1.1, 0.1N HCl with 2.75% NaCl at a rotation speed of 150 rpm

TABLE 9B
pH-Dissolution Profiles of RINVOQ (30 mg) of FIGS. 9-11

| | Formulation | | | |
|---|---|---|---|---|
| | RINVOQ (30 mg) | | | |
| | pH | | | |
| Time | pH 1.1^{~} | pH 1.1^{~} | pH 6.8^{#} | pH 6.8^{#} |
| (hr) | Release % | | | |
| 1 | 31 | 23-39 | 24 | 18-30 |
| 2 | 49 | 37-61 | 40 | 30-5 |
| 4 | 75 | 56-94 | 63 | 47-79 |
| 6 | 91 | 68-≥100 | 77 | 58-96 |
| 8 | 100 | 75-≥100 | 87 | 65-≥100 |
| 10 | 104 | 75-≥100 | 93 | 70-≥100 |
| 12 | 105 | 75-≥100 | 96 | 72-≥100 |
| 16 | 106 | 75-≥100 | 100 | 75-≥100 |
| 18 | 106 | 75-≥100 | 100 | 75-≥100 |
| 20 | 106 | 75-≥100 | 101 | 75-≥100 |

^{#}pH 6.8, 0.050M sodium phosphate buffer at a rotation speed of 150 rpm;
^{~}pH 1.1, 0.1N HCl with 2.75% NaCl at a rotation speed of 150 rpm

### Example 5. Effect of Hygroscopic Acidic pH Modifier in pH Dependent Polymer/Release Control Polymer Formulation

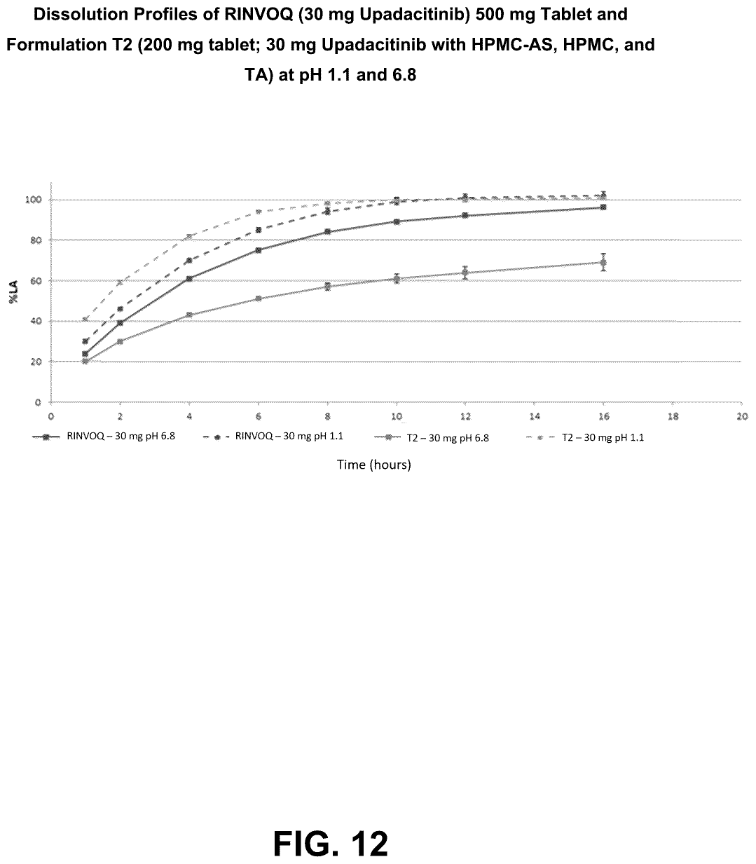

As is shown in FIG. 12, Formulation T2, containing 20% tartaric acid, while showing comparable dissolution to the dissolution profile of 30 mg RINVOQ tablet of Table 1 at pH 1.1, demonstrates a different dissolution profile at pH 6.8. The data suggests including higher amounts (e.g., 20% or greater) of a hygroscopic acidic pH modifier, such as tartaric acid, may result in a dissolution profile dissimilar to that of RINVOQ.

TABLE 10
HPMC-AS formulation with 20% Tartaric acid (T2)

| | | Amount | |
|---|---|---|---|
| | | per tablet | w/w |
| Component | Function | (mg)^{a} | (%) |
| Upadacitinib* | drug substance | 30.7 | 15.4% |
| Hydroxypropylmethyl | Release control | 40.0 | 20.0% |
| cellulose (HPMC K4M, | material | | |
| Hypromellose 2208) | | | |
| HPMC-acetate succinate | pH-dependent | 40.0 | 20.0% |
| (HPMCAS) | polymer | | |
| Tartaric acid | Hygroscopic | 40.0 | 20.0% |
| | Acidic pH | | |
| | modifier | | |
| Microcrystalline cellulose | filler | 20.2 | 10.1% |
| (MCC, Avicel PH102) | | | |
| Mannitol | | 20.0 | 10.0% |
| Hydroxypropyl cellulose | binder | 6.0 | 3.0% |
| (HPC) | | | |
| Colloidal silicon dioxide | glidant | 1.0 | 0.5% |
| Sodium stearyl fumarate | lubricant | 2.0 | 1.0% |
| Tablet Weight | | 200 | 100.0% |

^{a}Percents given based on the total tablet weight. Total percentage may not be 100% due to rounding.
*Upadacitinib was provided as Freebase Hydrate Form C, which is a hemihydrate, in an amount to deliver 30 mg of upadacitinib freebase equivalent.

In vitro dissolution rate of formulation T2 was determined using USP I method at a rotation speed of 150 rpm at 37° C. in 900 ml (1) pH 6.8, 0.025 M sodium phosphate buffer containing 2.75% NaCl and (2) pH 1.1, 0.1N HCl with 2.75% NaCl. Test results for T2 are provided in Table 11. The dissolution profile, compared to the dissolution profile of the RINVOQ (30 mg) tablet of Table 1 under the same dissolution conditions are also provided in FIG. 12.

TABLE 11
pH-Dissolution Profiles of T2 of FIG. 12

| | Formulation | |
|---|---|---|
| | T2 | |
| | pH | |
| Time | pH 1.1^{~} | pH 6.8^{\hat{ }} |
| (hr) | Release % | |
| 1 | 41 | 20 |
| 2 | 59 | 30 |
| 4 | 82 | 43 |
| 6 | 94 | 51 |
| 8 | 98 | 57 |
| 10 | 100 | 61 |
| 12 | 100 | 64 |
| 16 | 101 | 69 |

^{~}pH 1.1, 0.1N HCl with 2.75% NaCl at 150 rpm;
^{\hat{ }}pH 6.8, 0.025M sodium phosphate buffer containing 2.75% NaCl at 150 rpm.

### Example 6. Incorporation of Ion Exchange Resins (IER) as Release Rate Modifier

ER hydrophilic matrix tablets containing 15 mg or 30 mg Upadacitinib were prepared using hydroxypropyl methylcellulose as the rate controlling polymer and direct compression process. Compositions of tablet formulations and reference product (30 mg Rinvoq tablet) are provided in Table 1.

Formulation E1 was prepared as follows: 0.614 g of upadacitinib, 2.0 g of HPMC K750, 0.3 g of HPC EXF, 3.0 g of Amberlite IRP 69, 2.01 g of Avicel 102, 1.926 g of Pearlitol 100 SD, 0.05 g of Colloidal silicon dioxide were sieved through a 30 mesh screen and mixed for approximately 5 minutes in a Turbula blender (49 rpm). The powder blend was subsequently mixed for additional 2 min with 0.1 g of Magnesium stearate, followed by compression into 250 mg ovaloid shape tablets on a Carver press. Each tablet contains 15 mg of Upadacitinib (anhydrate form). The compositions of Formulation E1 and the reference product are provided in Table 12.

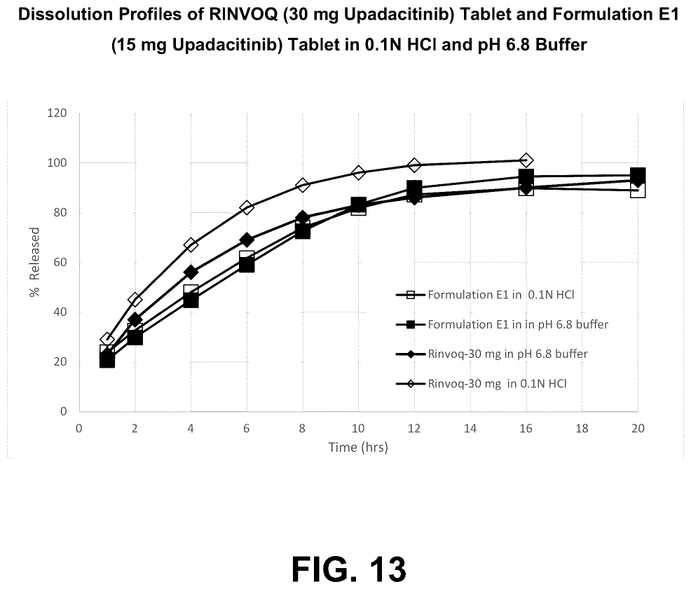

In vitro dissolution rate of formulation El was determined using USP 1 method at basket rotation speed of 100 rpm in 900 ml of (1) pH 6.8, 50 mM phosphate buffer and (2) 0.1N HCl with 50 mM sodium chloride at 37° C., respectively. In vitro dissolution rate of 30 mg Rinvoq tablets was determined using USP 1 method at basket rotation speed of 100 rpm in 900 ml of (1) pH 6.8, 50 mM phosphate buffer and (2) 0.1N HCl at 37° C., respectively. Dissolution test results provided in Table 13 and FIG. 13 show that similar extended release was obtained when compared to the reference product (30 mg Rinvoq). In addition, it offers advantages of near zero-order release, smaller tablet that is easier to swallow and minimized rate difference between 0.1N HCl and pH 6.8, i.e., pH-independent release despite of the significant solubility difference of the drug substance at pH 1.2 and pH 6.8.

TABLE 12
Compositions of Upadacitinib ER tablets of Example 6
(Formulation E1) and 30 mg Rinvoq tablet

| | 30 mg | 15 mg |
|---|---|---|
| | Rinvoq | Formulation |
| Material | Tablet | E1 |
| Upadacitinib* | 30.7 | 15.36 |
| HPMC K750 | NA | 50.00 |
| HPMC K4M | 96.0 | NA |
| HPC EXF | NA | 7.50 |
| Amberlite IRP69 | NA | 75.00 |
| Microcrystalline Cellulose | 147.2 | 50.25 |
| Mannitol (Pearlitol 100 SD) | 100.6 | 48.15 |
| Tartaric Acid | 96.0 | NA |
| Colloidal silicon dioxide | 2.4 | 1.25 |
| (Aerosil R972) | | |
| Magnesium stearate | 7.2 | 2.50 |
| Total Wt | 480 mg/Tab | 250 mg/tab |

*Hydrate form: Equal to 30 mg or 15 mg free base

TABLE 13
pH-Dissolution Profiles of Upadacitinib ER tablets of Example 6

| | Formulation | Formulation | Rinvoq- | Rinvoq-30 |
|---|---|---|---|---|
| Time | E1 in | E1 in | 30 mg in | mg in pH |
| (hr) | 0.1N HCl | pH 6.8 buffer | 0.1N HCl | 6.8 buffer |
| 1 | 23.9 | 20.7 | 29 | 23 |
| 2 | 32.6 | 29.8 | 45 | 37 |
| 4 | 48 | 44.8 | 67 | 56 |
| 6 | 61.7 | 59.0 | 82 | 69 |
| 8 | 73.9 | 72.5 | 91 | 78 |
| 10 | 81.8 | 83.2 | 96 | 83 |
| 12 | 87.2 | 89.9 | 99 | 86 |
| 16 | 89.8 | 94.5 | 101 | 90 |
| 20 | 88.9 | 95.0 | 101 | 93 |

### Example 7. Incorporation of an Anionic Polymer and a Basic pH Modifier

Three formulations containing either HPMCP (HP-55) or Na₂CO₃ or a combination of HPMC HP-55 and Na₂CO₃ as release rate modifiers were prepared in this study. All three formulations of the ER hydrophilic matrix tablets were prepared using direct compression process. Materials listed in Table 14 were weighed and sieved through a 30 mesh screen prior to blending. Drug and all excipients except magnesium stearate (MgSt) was first blended for 5 min@49 rpm in a turbula blender, this was followed by adding MgSt and blended for an additional 2 min. The final blend was compressed into a 200 mg tablet using oval-shaped tooling at ˜3000 lbs. on a Carver press. Each tablet contains 30 mg of upadacitinib (anhydrate form).

TABLE 14
Compositions of Upadacitinib ER tablets of Example 7

| | Formulation E2 | | Formulation E3 | | Formulation E4 | |
|---|---|---|---|---|---|---|
| Ingredient | mg | % | mg | % | mg | % |
| Upadacitinib* | 30.7 | 15.4 | 30.7 | 15.4 | 30.7 | 15.4 |
| HPMC, Methocel | 30.0 | 15.0 | 30.0 | 15.0 | 30.0 | 15.0 |
| K4M | | | | | | |
| Hydroxypropyl | 6.0 | 3.0 | 6.0 | 3.0 | 6.0 | 3.0 |
| cellulose | | | | | | |
| HPMCP HP-55 | 40.0 | 20.0 | NA | NA | 40.0 | 20.0 |
| Na2CO3 | NA | NA | 20.0 | 10.0 | 20.0 | 10.0 |
| monohydrate | | | | | | |
| Microcrystalline | 50.2 | 25.1 | 70.2 | 35.1 | 30.2 | 15.1 |
| cellulose | | | | | | |
| Mannitol | 40.0 | 20.0 | 40.0 | 20.0 | 40.0 | 20.0 |
| (Pearlitol | | | | | | |
| 100 SD) | | | | | | |
| Colloidal | 1.0 | 0.5 | 1.0 | 0.5 | 1.0 | 0.5 |
| silicon | | | | | | |
| dioxide | | | | | | |
| Magnesium | 2.0 | 1.0 | 2.0 | 1.0 | 2.0 | 1.0 |
| stearate | | | | | | |
| (MgSt) | | | | | | |
| Total | 200.0 | 100.0 | 200.0 | 100.0 | 200.0 | 100.0 |

*Hydrate form: Equal to 30 mg free base

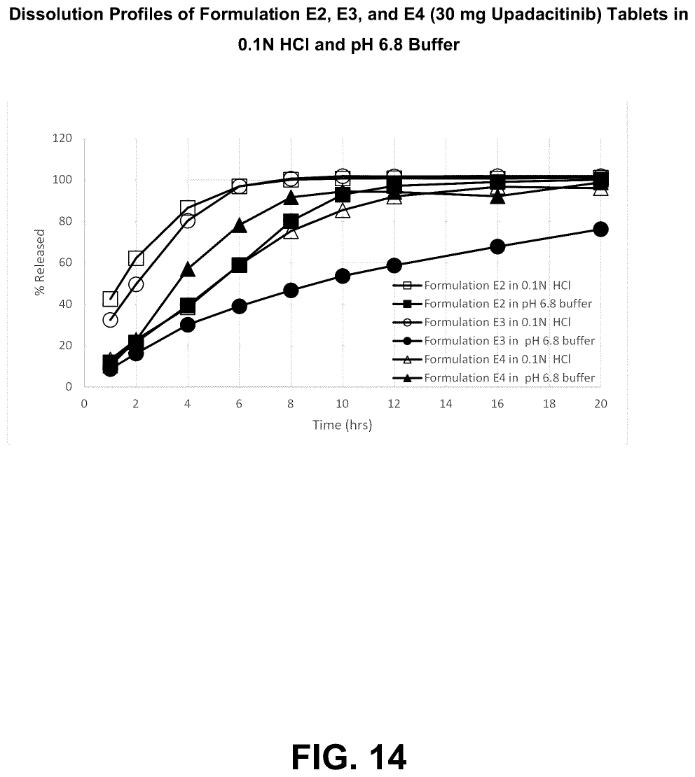

In vitro dissolution rates were determined using USP 1 method at basket rotation speed of 100 rpm in 900 ml of (1) 0.1N HCl; (2) pH 6.8, 50 mM phosphate buffer at 37° C., respectively. At least three tablets were used under each test condition. Dissolution results provided in Table 15 and FIG. 14 show that extended drug release was obtained for all three formulations. pH-dependent dissolution was observed for Formulations E2 and E3. However, pH dependency of the release rate was significantly reduced through a combined use of HPMCP HP-55 and Na₂CO₃ as release rate modifiers, an improvement over that of Rinvoq tablets.

TABLE 15
pH-Dissolution Profiles of Upadacitinib ER tablets of Example 7

| | E2 in | E2 in | E3 in | E3 in | E4 in | E4 in |
|---|---|---|---|---|---|---|
| | 0.1N | pH 6.8 | 0.1N | pH 6.8 | 0.1N | pH 6.8 |
| time (hrs) | HCl | buffer | HCl | buffer | HCl | buffer |
| 1 | 41.4 | 11.6 | 31.6 | 8.4 | 12.9 | 10.0 |
| 2 | 60.7 | 21.0 | 48.4 | 15.7 | 22.2 | 22.7 |
| 4 | 84.5 | 38.4 | 78.4 | 29.3 | 37.5 | 53.3 |
| 6 | 94.6 | 57.5 | 94.6 | 38.0 | 57.5 | 76.3 |
| 8 | 97.7 | 78.2 | 98.2 | 45.5 | 73.5 | 88.0 |
| 10 | 98.4 | 90.6 | 99.4 | 52.3 | 83.3 | 89.8 |
| 12 | 98.4 | 94.8 | 99.3 | 57.3 | 88.8 | 90.3 |
| 16 | 98.3 | 96.6 | 99.3 | 66.2 | 93.0 | 89.9 |
| 20 | 98.7 | 97.7 | 99.4 | 74.4 | 93.6 | 91.5 |

### Example 8. Study of the Effect of Manufacturing Process on Drug Release for Tablets Containing an Anionic Polymer and a Basic pH Modifier

A wet granulation process was also used to prepare 30 mg Upadacitinib tablets containing both HPMCP HP-55 and Na₂CO₃ as release rate modifiers. Tablet formulation E5 was prepared using the same composition as formulation E4 and wet granulation process. 15.36 g of upadacitinib, 15 g of HPMC K4M, 20 g of HPMCP HP-55, 10 g of Sodium carbonate monohydrate, 15.10 g of Avicel 101 were first sieved, respectively, and dry mixed in a bench-top high-speed mixer followed by granulation using ˜32 g of water. The wet granules were vacuum dried overnight @60° C. The dry granules were sieved through a 30 mesh screen and blended with the extragranular excipients of Table 16 for approximately 5 minutes on a Turbula blender (49 rpm). The powder blend was subsequently mixed with magnesium stearate for 2 min and subsequently compressed into 200 mg ovaloid shape tablets on a Carver press. Each tablet contains 30 mg of Upadacitinib (anhydrate form).

TABLE 16
Compositions of Upadacitinib ER tablets of Example 8

| | | |
|---|---|---|
| | mg per | Formulation |
| Formulation E5 | tablet | % (w/w) |

Intergranular

| | | |
|---|---|---|
| Upadacitinib* | 30.7 | 15.36% |
| HPMC K4M | 30.0 | 15.00% |
| HPMCP HP-55 | 40.0 | 20.00% |
| Sodium carbonate monohydrate | 20.0 | 10.00% |
| Microcrystalline cellulose | 30.2 | 15.10% |

Extragranular

| | | |
|---|---|---|
| Colloidal silicon dioxide | 1.00 | 0.5% |
| Mannitol (Pearlitol 100 SD) | 40.0 | 20.00% |
| HPC (Klucel EXF) | 6.00 | 3.00% |
| Magnesium Stearate | 2.00 | 1.00% |
| Total Wt | 200 mg | 100.00% |

*Hydrate form: Equal to 30 mg free base

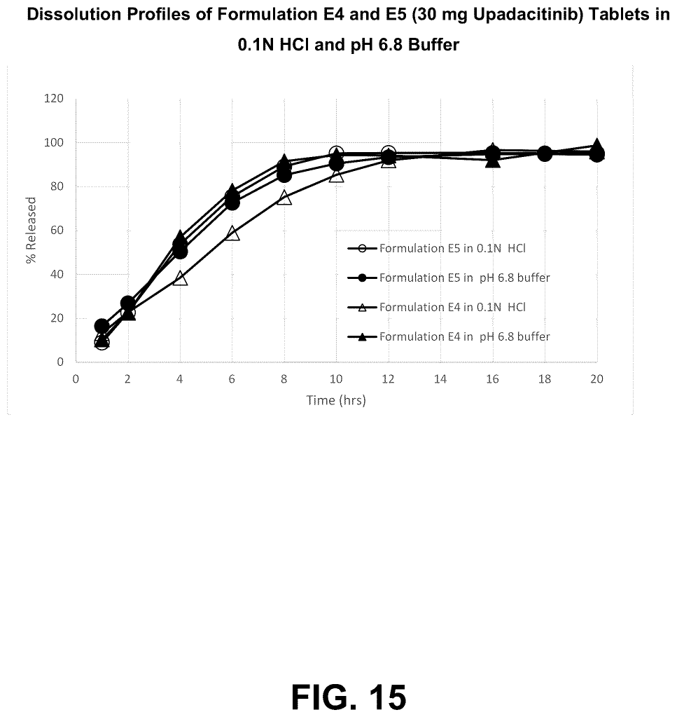

In vitro dissolution rates of Formulation E5 was determined using USP 1 method at 100 rpm in 900 ml of (1) 0.1N HCl; (2) pH 6.8, 50 mM phosphate buffer at 37° C., respectively. At least three tablets were used under each test condition. Dissolution test results of tablets made with wet granulation and direct compression processes are provided in Table 17 and FIG. 15, respectively. These data show that pH-dependency of Upadacitinib release from hydrophilic matrix tablets containing dual release rate modifiers can be further minimized using wet granulation process, resulting in pH-independent release.

TABLE 17
pH-Dissolution Profiles of Upadacitinib ER tablets of Example 8

| | Formulation | Formulation | Formulation | Formulation |
|---|---|---|---|---|
| time | E4 in | E4 in pH | E5 in | E5 in pH |
| (hrs) | 0.1N HCl | 6.8 buffer | 0.1N HCl | 6.8 buffer |
| 1 | 12.9 | 10.0 | 16.4 | 8.9 |
| 2 | 22.2 | 22.7 | 26.8 | 22.7 |
| 4 | 37.5 | 53.3 | 50.4 | 53.7 |
| 6 | 57.5 | 76.3 | 72.6 | 75.4 |
| 8 | 73.5 | 88.0 | 85.2 | 89.2 |
| 10 | 83.3 | 89.8 | 90.5 | 95.1 |
| 12 | 88.8 | 90.3 | 93.4 | 95.3 |
| 16 | 93.0 | 89.9 | 94.7 | 95.3 |
| 20 | 93.6 | 91.5 | 94.8 | 95.1 |

### Example 9. pH-Independent Release of Upadacitinib from Hydrophilic Matrices by Modulating Release Surface Area

Upadacitinib ER hydrophilic matrix tablets (Formulation E6) with reduced pH-dependency similar to that of Rinvoq tablets had been prepared using acidic release rate modifier and direct compression process. To further decrease pH-dependency of the drug release or enable pH-independent release, a barrier layer was applied to the partial tablet surface using pH-dependent polymer. Two barrier formulations were used: (1) repeated application of a pH-dependent polymer coating solution on one side of the tablet surface (Formulation E7) (2) application of a pH-dependent layer on one side of the tablet surface by compression coating (Formulation E8).

The compositions and preparation of the reference and test tablets are described in the following sections.

### Part A: Reference ER Core Tablet Preparation (Formulation E6)

Upadacitinib ER hydrophilic matrix tablets, Formulation E6, (Lot: S-211004-korecsa-073) were prepared using a direct compression process. Materials listed in Table 18 were weighted and sieved through a 30 mesh screen prior to blending, respectively. Drug and all excipients except sodium stearyl fumarate was first blended for 5 min @49 rpm in a turbula blender, this was followed by adding sodium stearyl fumarate and blending for an additional 2 min. The final blend was compressed into a monolithic 200 mg tablet (reference tablets) using oval-shaped tooling under ˜3000 lbs force on a Carver press. Each tablet contains 30 mg of upadacitinib (anhydrate form).

### Part B: Preparation of the Film Coating Solution

The 5% (w/w) coating solution was prepared by mixing 4.5 g of HPMCAS LG and 0.5 g of PEG 3350 in acetone/water (90/10) with stirring until complete dissolution.

### Part C: Preparation of the Compression Coating Layer Blend

9.2 g of HPMCAS (LG), 0.75 g of Fastflo lactose were sieved through a 35 mesh screen and mixed for approximately 5 minutes in a Turbula blender (49 rpm). The powder blend was subsequently blended for an additional 2 min with 0.05 g of magnesium stearate.

### Part D: Preparation of Partially Coated ER Tablets Using Solvent Process (Formulation E7)

Application of barrier layer was carried out as follows: Each tablet was first mounted on the tip of a tweezer. Coating solution was applied on one side of the tablet surface by iterative dipping and 4-minute air dry operations. The process is repeated for 10 times. Upon completion of the dip coating process, the tablets were placed in an oven at 40° C. for at least 24 hours prior to dissolution testing. The total weight gain after coating/drying for each tablet is approximately 8 mg.

### Part E: Preparation of Partially Coated ER Tablets Using Compression Coating Process (Formulation E8)

The bilayer tablet of Formulation E8 was prepared on a Carver press as follows: 200 mg of Formulation E6 blend was loaded into oval-shaped tooling die cavity, followed by applying a low tamping force with the upper punch, this was followed by adding 40 mg of the compression coat layer blend, and subsequently compressing at ˜3000 lbs. Each tablet contains 30 mg of upadacitinib (anhydrate form).

TABLE 18
Compositions of Upadacitinib ER tablets
of Example 9 Formulations E6

| | Formulation E6 | |
|---|---|---|
| Ingredient | mg | % |
| Upadacitinib* | 30.7 | 15.4 |
| HPMC, Methocel K4M | 50.0 | 25.0 |
| Hydroxypropyl cellulose | 6.0 | 3.0 |
| Fumaric acid | 60.0 | 30.0 |
| Microcrystalline cellulose | 20.3 | 10.2 |
| Mannitol (Pearlitol 100 SD) | 30.0 | 15.0 |
| Colloidal silicon dioxide | 1.0 | 0.5 |
| Sodium stearyl fumarate (SSF) | 2.0 | 1.0 |
| Total | 200.0 | 100.0 |

*Hydrate form: Equal to 30 mg free base

### Part F: Dissolution Testing of Formulations E6, E7 and E8

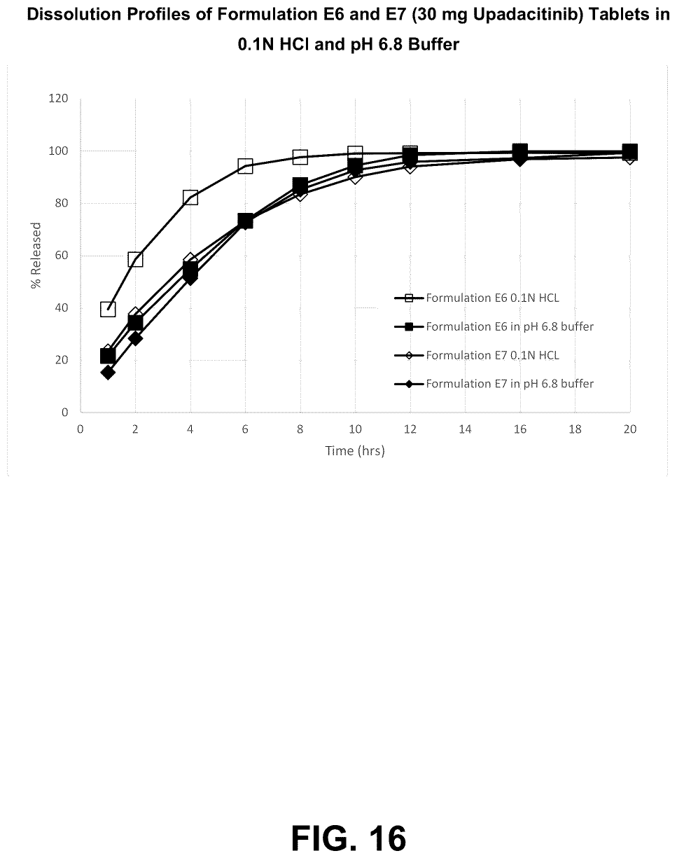

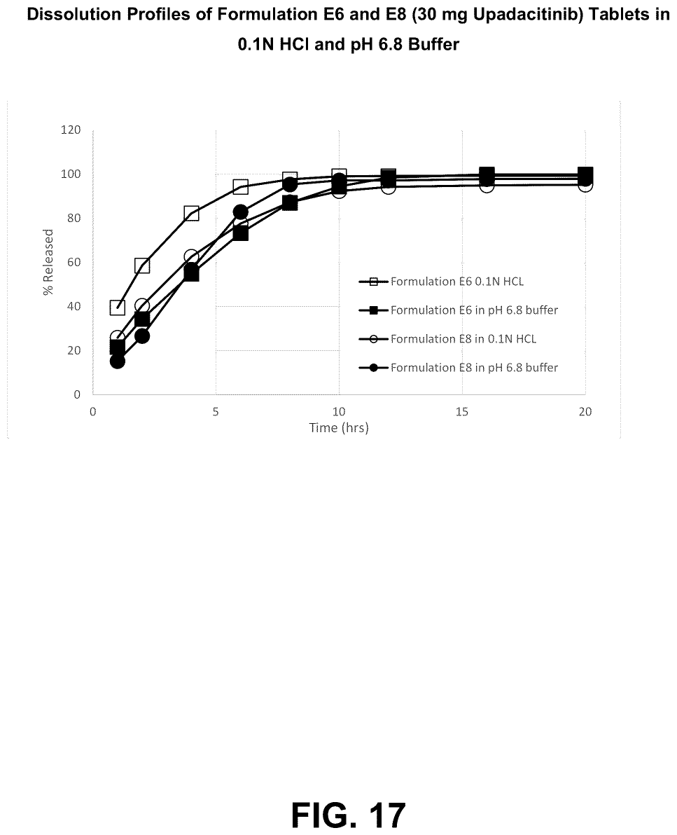

In vitro dissolution rates of test and reference tablets were determined using USP 1 method at 150 rpm in 900 ml of (1) pH 6.8, 25 mM phosphate buffer with 2.7% NaCl; and (2) 0.1N HCl at 37° C. respectively. At least three tablets were used under each testing condition. Dissolution test results provided in Table 19. FIG. 16 shows comparative dissolution profiles of Formulations E6 and E7; and FIG. 17 shows comparative dissolution profiles of Formulations E6 and E8. Results from these figures indicate moderate pH-dependency of the release rates of the reference tablets can be essentially eliminated by applying pH-dependent barrier layer onto the partial surface of the tablet using either solvent based coating and compression coating processes.

TABLE 19
pH-Dissolution Data of Upadacitinib ER tablets of Example 9

| | Formulations | Formulations | Formulations | Formulations | Formulations | Formulations |
|---|---|---|---|---|---|---|
| | E6 in | E6 in | E7 in | E7 in | E8 in | E8 in |
| Time | 0.1N | pH 6.8 | 0.1N | pH 6.8 | 0.1N | pH 6.8 |
| (hr) | HCL | buffer | HCL | buffer | HCl | buffer |
| 1 | 39.4 | 21.6 | 23.4 | 15.3 | 25.3 | 14.9 |
| 2 | 58.5 | 34.3 | 37.6 | 28.3 | 39.4 | 26.0 |
| 4 | 82.2 | 54.8 | 58.4 | 51.3 | 61.1 | 55.5 |
| 6 | 94.2 | 73.3 | 73.2 | 72.6 | 75.9 | 81.0 |
| 8 | 97.6 | 87.0 | 83.4 | 85.2 | 85.3 | 93.1 |
| 10 | 99.0 | 94.4 | 90.0 | 92.6 | 90.2 | 95.0 |
| 12 | 99.1 | 98.4 | 94 | 95.8 | 92.1 | 94.9 |
| 16 | 99.3 | 99.9 | 96.8 | 97.2 | 92.8 | 95.6 |
| 20 | 99.2 | 99.8 | 97.5 | 99.3 | 93.0 | 95.6 |

### Example 10. pH-Independent Upadacitinib Release Using Osmotic Pump Tablets

Upadacitinib ER tablets were prepared using an osmotic pump delivery system. The final dosage form consists of either a single layer, or bilayer or a triple layer core tablet containing osmotic agents (osmogent) coated with a semi-permeable membrane. An orifice was formed by mechanical drilling on the tablet surface of the drug layer to facilitate drug release.

Formulations and processes for preparing different osmotic pump tablets are summarized in the following sections.

### Part I: Preparation of the Drug Layer and Push Layer Blends

Drug layer blend A: 7.68 g of upadacitinib, 21.32 g of Sorbitol (Neosorb P60W), 20 g of polyethylene oxide (polyox WSR N-80N), 20 g of Sodium Chloride (milled), 25 g fumaric acid, 5 g of HPC (Klucel EXF) were sieved through a 30 mesh screen, mixed for approximately 5 minutes in a Turbula blender (49 rpm). The powder blend was subsequently mixed for an additional 2 min with 1 g of magnesium stearate.

Drug layer blend B: 0.768g of upadacitinib, 2.132 g of Sorbitol (Neosorb P60W), 2.0 g of polyethylene oxide (polyox WSR N-80N), 2.0 g of Sodium Chloride (milled), 2.5 g of lactose, 0.5 g of HPC (Klucel EXF) were sieved through a 30 mesh screen, mixed for approximately 5 minutes in a Turbula blender (49 rpm). The powder blend was subsequently mixed for an additional 2 min with 0.1 g of Magnesium stearate.

Push layer blend: 60 g of HEC (Natrosol, 250 HX), 20 g of Sorbitol (Neosorb P60W), 16 g of Sodium Chloride (milled), 3.0 g of HPC (Klucel EXF), were sieved through a 30 mesh screen, mixed for approximately 5 minutes in a Turbula blender (49 rpm). The powder blend was subsequently mixed for an additional 2 min with 1 g of magnesium stearate.

The formulation compositions of drug and push layers are shown in Table 20.

TABLE 20
The formulation compositions of drug and push layers

| | Formulation | | | |
|---|---|---|---|---|
| | E9, E10, E11 | | E12 | |
| Ingredient | (mg/unit) | % | (mg/unit) | % |

Drug Layer

| | | | | |
|---|---|---|---|---|
| Upadacitinib* | 11.52 | 7.68 | 11.52 | 7.68 |
| Sorbitol (Neosorb P60 W) | 31.98 | 21.32 | 31.98 | 21.32 |
| Fumaric acid | 37.5.0 | 25.00 | — | — |
| Lactose | — | — | 37.50 | 25.00 |
| Polyethylene oxide, | 30.00 | 20.00 | 30.00 | 20.00 |
| (POLYOX ™ WSR N-80 N) | | | | |
| NaCl (milled) | 30.00 | 20.00 | 30.00 | 20.00 |
| HPC (Klucel EXF) | 7.50 | 5.00 | 7.50 | 5.00 |
| Colorant (blue dye) | Trace | Trace | Trace | Trace |
| Magnesium Stearate (MgSt) | 1.50 | 1.00 | 1.50 | 1.00 |
| Sub-weight | 150.00 | 100.00 | 150.00 | 100.00 |

Push layer

| | | | | |
|---|---|---|---|---|
| HEC (Natrosol, HX) | 90.00 | 60.00 | 90.00 | 60.00 |
| Natrosol 250 HX | | | | |
| Sorbitol (Neosorb P60 W) | 30.00 | 20.00 | 30.00 | 20.00 |
| Sodium Chloride (milled) | 24.00 | 16.00 | 24.00 | 16.00 |
| HPC (Klucel EXF) | 4.50 | 3.00 | 4.50 | 3.00 |
| Magnesium Stearate (MgSt) | 1.50 | 1.00 | 1.50 | 1.00 |
| Total weight in mg and % | 300.00 | 100.00 | 300.00 | 100.00 |

*Hydrate form: Equal to 11.25 mg free base

### Part II: Preparation of Monolithic Layer Tablet (Formulation E9)

Monolithic layer core tablet was prepared on a Carver Press as follows: 150 mg drug layer blend and 150 mg of push layer blend were weighed and mixed thoroughly and then loaded into the die cavity using an 8 mm round convex tooling and compressed into the final tablet with approximately 3000 lbs force. The final core tablet weight was 300 mg which contains 11.25 mg of Upadacitinib (anhydrate form).

### Part III: Preparation of Bilayer Tablet A (Formulation E10)

Bilayer core tablet was prepared on the Carver Press as follows: 150 mg of drug layer A and push layer blends were weighed, separately. Drug layer blend was first loaded into the die cavity using an 8 mm round convex tooling, followed by a gentle tamping with the upper punch, push layer was then added on top of the drug layer, a final compression force of ˜3000 pounds was applied to form the bilayer tablet. The final core tablet weight was 300 mg which contains 11.25 mg of Upadacitinib (anhydrate form).

### Part IV: Preparation of Triple Layer Tablet (Formulation E11)

Triple core tablet was prepared on the Carver Press as follows: 150 mg of drug layer blend, 150 mg of push layer blends and 25 mg separation layer (ethylcellulose) were weighed separately. The drug layer blend was first loaded into the die cavity using a 6 mm round convex tooling, followed by a gentle tamping with the upper punch, the separation layer was then added on top of the drug layer followed by another gentle tamping, the push layer was added lastly on top of the separation layer. A final compression force of ˜3000 pounds was applied to form a triple layer tablet. The final core tablet weight was 325 mg which contains 11.25 mg of Upadacitinib (anhydrate form).

### Part V: Preparation of Bilayer Tablet B (Formulation E12)

Bilayer core tablet was prepared on the Carver Press as follows: 150 mg of drug layer B and push layer blends were weighed, separately. Drug layer blend was first loaded into the die cavity using an 8 mm round convex tooling, followed by a gentle tamping with the upper punch, the push layer was then added on top of the drug layer, a final compression force of ˜3000 pounds was applied to form the bilayer tablet. The final core tablet weight was 300 mg which contains 11.25 mg of Upadacitinib (anhydrate form).

### Part VI: Preparation of the Coating Solution

Solvent for coating solution was prepared by weighing 98 g of acetone and 2 g of water into a beaker and mixing well. A 5% (w/w) dip coating solution was prepared by slowly adding 5 g of Opadry CA (Colorcon, fully formulated osmotic coating system 500F 190012 Clear) into the 95 g of the 98/2 Acetone/water solution, mixing until the solution is clear.

### Part VII: Preparation of Coated Tablets for Dissolution Tests

The core tablets of Formulations E9, E10, E11and E12 were first mounted onto the sharp tips of the forceps. This is followed by dipping the entire tablet in the coating solution followed by air dry for 4 minutes. This operation is repeated for 25 times. Upon completing the application of the coating film, the tablets were transferred to a 40° C. oven, and dried for at least 24 hours. After drying, these tablets were weighed to calculate the coating weight gain. The average post drying weight gain for Formulations E9, E10, E11 and E12 were 46.5 mg, 41.7 mg,46.7 mg and 44.05 mg, respectively. Orifice sizes of 0.5 mm (Formulation E9), 1.6 mm (Formulation E10 and Formulation E12) and 2.0 mm (Formulation E11) were mechanically drilled into the drug layer side, around the tips of the forceps used to mount the tablets.

### Part VIII: Dissolution of Coated Tablets of Formulation E9, Formulation E10, Formulation E11 and Formulation E12

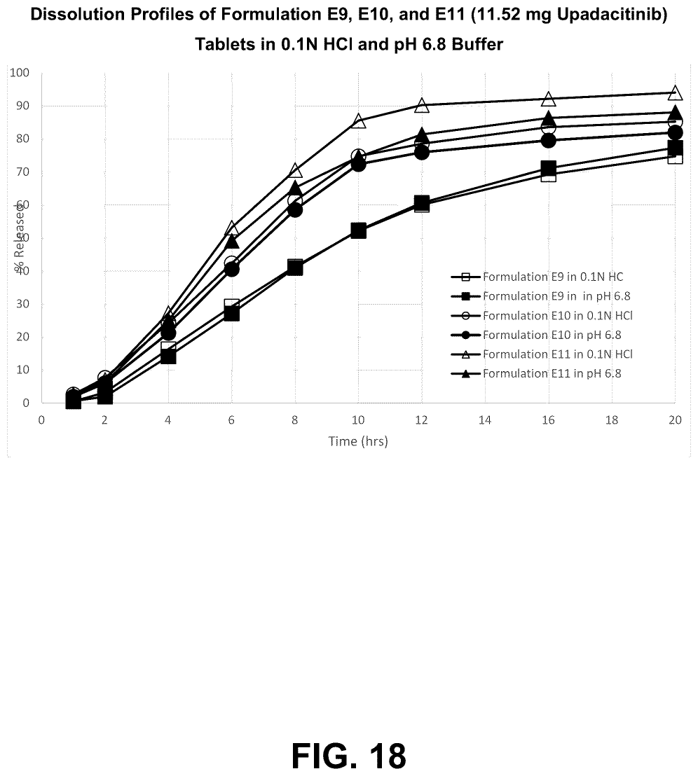

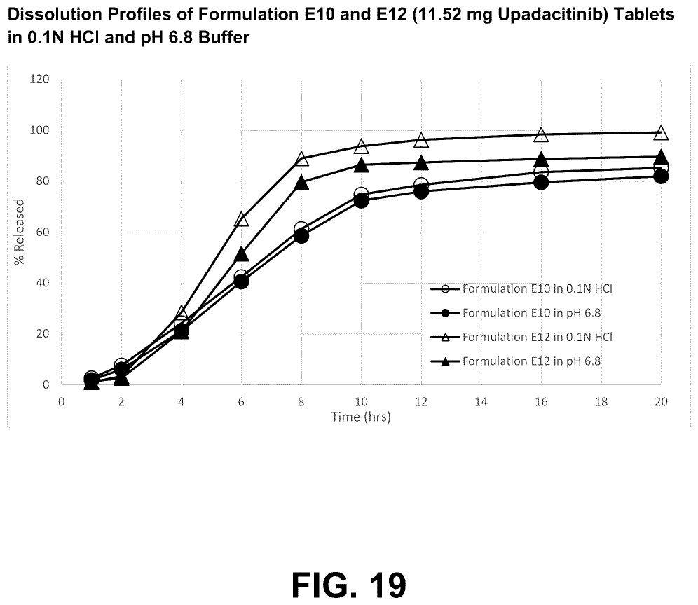

In vitro dissolution rates of the osmotic pump tablets were determined using USP 1 method at 100 rpm in 900 ml of (1) pH 6.8, 50 mM phosphate buffer and (2) pH 1.2, 0.1N HCl at 37° C., respectively. At least three tablets were used under each test condition. Dissolution test results are provided in Table 21. FIG. 18 shows that pH-independent and near zero-order drug release was obtained for all three formulations. FIG. 19 shows that pH-independent and near zero-order drug release can be achieved with same product design independent of presence of acidic release rate modifiers (such as, fumaric acid).

TABLE 21
pH-Dissolution Profiles of Upadacitinib Osmotic pump tablets of Example 10

| | E9 in | E9 in | E10 in | E10 in | E11 in | E11 in | E12 in | E12 in |
|---|---|---|---|---|---|---|---|---|
| Time | 0.1N | pH 6.8 | 0.1N | pH 6.8 | 0.1N | pH 6.8 | 0.1N | pH 6.8 |
| (hr) | HCl | buffer | HCl | buffer | HCl | buffer | HCl | buffer |
| 1 | 0.5 | 0.6 | 2.6 | 1.9 | 2.1 | 2.5 | 1.0 | 1.3 |
| 2 | 3.2 | 1.9 | 7.6 | 6.0 | 7.0 | 6.1 | 3.3 | 2.6 |
| 4 | 16.3 | 14.1 | 24.2 | 21.2 | 27.2 | 25.0 | 28.4 | 20.9 |
| 6 | 29.2 | 27.1 | 42.3 | 40.5 | 53.1 | 49.1 | 65.2 | 51.5 |
| 8 | 41.3 | 40.9 | 61.2 | 58.5 | 70.5 | 65.2 | 88.9 | 79.6 |
| 10 | 52.1 | 52.3 | 74.7 | 72.3 | 85.5 | 74.5 | 93.7 | 86.4 |
| 12 | 60.0 | 60.6 | 78.5 | 75.9 | 90.2 | 81.3 | 96.2 | 87.3 |
| 16 | 69.2 | 71.1 | 83.5 | 79.5 | 92.1 | 86.3 | 98.3 | 88.7 |

### OTHER EMBODIMENTS

This application refers to various issued patent, published patent applications, journal articles, and other publications, each of which is incorporated herein by reference.

The foregoing has been described of certain non-limiting embodiments of the present disclosure. Those of ordinary skill in the art will appreciate that various changes and modifications to this description may be made without departing from the spirit or scope of the present disclosure, as defined in the following claims.

What is claimed is:

1. An extended release solid dosage form comprising upadacitinib, or a pharmaceutically acceptable salt thereof, at least one pH dependent polymer and at least one release control material.

2. The extended release solid dosage form of claim 1, wherein the at least one pH dependent polymer and the at least one release control material comprise a matrix system containing upadacitinib or a pharmaceutically acceptable salt thereof.

3. The extended release solid dosage form of any one of claims 1-2, wherein the dosage form does not comprise an enteric film coat.

4. The extended release solid dosage form of any one of claims 1-3, wherein the at least one pH dependent polymer is an enteric polymer or an anionic polysaccharide.

5. The extended release solid dosage form of any one of claims 1-4, wherein the at least one pH dependent polymer is hydroxypropylmethyl cellulose acetate succinate (HPMCAS) or alginic acid.

6. The extended release solid dosage form of any one of claims 1-5, wherein the at least one pH dependent polymer is present in an amount from about 20% to about 40% w/w and the at least one release control material is present in an amount from about 30% to about 60% w/w of the solid dosage form.

7. The extended release solid dosage form of any one of claims 1-6, further comprising a basic pH modifier.

8. The extended release solid dosage form of claim 7, wherein the basic pH modifier is sodium carbonate, meglumine, or tribasic sodium phosphate dodecahydrate.

9. An extended release solid dosage form comprising upadacitinib, or a pharmaceutically acceptable salt thereof, at least one release rate modifier and at least one release control material, wherein
(a) the at least one release rate modifier comprises an ion exchange resin, or
(b) the at least one release rate modifier comprises a basic pH modifier and the extended release solid dosage form further comprises an anionic polymer.

10. The extended release solid dosage form of claim 9, wherein the at least one release rate modifier is an ion exchange resin.

11. The extended release solid dosage form of claim 10, wherein the ion exchange resin comprises a sulfonated copolymer comprising styrene and divinylbenzene.

12. The extended release solid dosage form of claim 10, wherein upadacitinib, or a pharmaceutically acceptable salt thereof, and the ion exchange resin form an upadacitinib-ion exchange resin complex, said upadacitinib-ion exchange resin complex comprising upadacitinib, or a pharmaceutically acceptable salt thereof, bound to the ion exchange resin.

13. The extended release solid dosage form of claim 9, wherein the at least one release rate modifier is a basic pH modifier and the extended release solid dosage form further comprises an anionic polymer.

14. The extended release solid dosage form of claim 13, wherein the basic pH modifier is present in an amount from about 5% to about 20% w/w.

15. The extended release solid dosage form of any one of claims 13-14, wherein the basic pH modifier is sodium carbonate, meglumine, or tribasic sodium phosphate dodecahydrate.

16. An extended release solid dosage form comprising:
a core comprising upadacitinib, or a pharmaceutically acceptable salt thereof, at least one release rate modifier and at least one release control material; and
a barrier layer partially covering the core.

17. The extended release solid dosage form of claim 16, wherein the at least one release rate modifier is an acidic pH modifier.

18. The extended release solid dosage form of any of claims 16-17, wherein barrier layer comprises a pH-dependent polymer.

19. The extended release solid dosage form of claim 18, wherein the pH-dependent polymer is hydroxypropylmethyl cellulose acetate succinate (HPMCAS).

20. The extended release solid dosage form of any of claims 1-19, wherein the dosage form comprises less than 10% by weight of a hygroscopic acidic pH modifier.

21. The extended release solid dosage form of any of claims 1-19, wherein the dosage form is substantially free of a hygroscopic acidic pH modifier.

22. The extended release solid dosage form of any of claims 20-21, wherein the hygroscopic acidic pH modifier is an organic acid.

23. The extended release solid dosage form of claim 22, wherein the hygroscopic organic acid is selected from the group consisting of tartaric acid, citric acid, and maleic acid.

24. The extended release solid dosage form of any one of claims 1-23, wherein the at least one release control material is a release control polymer.

25. The extended release solid dosage form of any one of claims 1-24, wherein the at least one release control polymer is selected from the group consisting of hydroxypropylmethyl cellulose (HPMC), hydroxypropyl cellulose, hydroxyethyl cellulose, a copolymer of acrylic acid crosslinked with a polyalkenyl polyether (Carbopol), non-ionic homopolymer of ethylene oxide (Polyox), a water soluble natural gum of a polysaccharide, crosslinked starch, polyvinyl acetate, polyvinylpyrrolidone, and combinations thereof.

26. The extended release solid dosage form of any one of claims 1-25, wherein the solid dosage form further comprises one or more additional excipients selected from the group consisting of a filler, a binder, a glidant, a lubricant, a film coat, and combinations thereof; wherein the one or more additional excipients are present in an amount less than 50% w/w of the solid dosage form.

27. An extended release solid dosage form comprising:
a core comprising upadacitinib, or a pharmaceutically acceptable salt thereof;
a semi-permeable membrane covering the core, wherin the semi-permeable membrane is permeable to aqueous fluid and substantially impermeable to upadacitinib; and
at least one drug delivery orifice that provides a passageway such that upadacitinib, or the pharmaceutically acceptable salt thereof, can be released from the core into an environment external to the extended release solid dosage form.

28. The extended release solid dosage form of claim 27, wherein the core further comprises an osmogent.

29. The extended release solid dosage form of claim 28, wherein the osmogent comprises a water soluble salt of an inorganic acid, an osmotic polymer, a carbohydrate, or a combinations thereof.

30. The extended release solid dosage form of any one of claims 27-29, wherein the core is single-layered or multi-layered.

31. The extended release solid dosage form of any one of claims 27-29, wherein the core comprises a push layer.

32. The extended release solid dosage form of claim 31, wherein the push layer comprises an osmotic polymer, preferably, hydroxyethylcellulose (HEC).

33. The extended release solid dosage form of any one of claims 27-32, wherein the semi-permeable membrane comprises cellulose acetate (CA).

34. A stable solid dosage form comprising upadacitinib or a pharmaceutically acceptable salt thereof, at least one pH dependent polymer, and at least one release control material;
wherein no more than 0.2% w/w of a upadacitinib degradation product is present in the solid dosage form at an initial timepoint and no more than 0.5% w/w of the upadacitinib degradation product is present in the solid dosage form at a post-storage timepoint;
wherein the initial and post-storage timepoints are separated by at least 3 months, at least 6 months, at least 9 months, at least 12 months, at least 18 months, at least 24 months, at least 30 months, or at least 36 months during which the composition is at 25° C.±2° C. and 60%±5% relative humidity; and
wherein the upadacitinib degradation product is (3S,4R)-3-ethyl-4-(3-(hydroxymethyl)-3H-imidazo[1,2-a]pyrrolo[2,3-e]pyrazin-8-yl)-N-(2,2,2-trifluoroethyl)pyrrolidine-1-carboxamide.

35. The stable solid dosage form of claim 34, wherein the at least one pH dependent polymer is an enteric polymer or an anionic polysaccharide.

36. The stable solid dosage form of claim 35, wherein the at least one pH dependent polymer is hydroxypropylmethyl cellulose acetate succinate (HPMCAS) or alginic acid.

37. The stable solid dosage form of any one of claims 34-36, wherein from about 68% to about 100% of upadacitinib is released from the solid dosage form in about 6 hours using USP I method at a rotation speed of 150 rpm in 900 ml of pH 1.1, 0.1N HCl at 37° C. and/or from about 75% to about 100% of upadacitinib is released from the solid dosage form in about 8 hours using USP I method at a rotation speed of 150 rpm in 900 ml of pH 1.1, 0.1N HCl at 37° C.

38. The stable solid dosage form of claim 37, wherein from about 56% to about 94% of upadacitinib is released from the solid dosage form in about 4 hours using USP I method at a rotation speed of 150 rpm in 900 ml of pH 1.1, 0.1N HCl at 37° C.

39. The stable solid dosage form of any one of claims 34-36, wherein from about 51% to about 85% of upadacitinib is released from the solid dosage form in about 6 hours using USP I method at a rotation speed of 150 rpm in 900 ml of pH 6.8, 0.025 M sodium phosphate buffer containing 2.75% sodium chloride at 37° C. and/or from about 59% to about 99% of upadacitinib is released from the solid dosage form in about 8 hours using USP I method at a rotation speed of 150 rpm in 900 ml of pH 6.8, 0.025 M sodium phosphate buffer containing 2.75% sodium chloride at 37° C.

40. The stable solid dosage form of claim 39, wherein from about 41% to about 68% of upadacitinib is released from the solid dosage form in about 4 hours using USP I method at a rotation speed of 150 rpm in 900 ml pH 6.8, 0.025 M sodium phosphate buffer containing 2.75% sodium chloride at 37° C.

41. The stable solid dosage form of any one of claims 34-36, wherein from about 60% to about 100% of upadacitinib is released from the solid dosage form in about 6 hours using USP I method at a rotation speed of 150 rpm in 900 ml of pH 6.8, 0.025 M sodium phosphate buffer at 37° C. and/or from about 67% to about 100% of upadacitinib is released from the solid dosage form in about 8 hours using USP I method at a rotation speed of 150 rpm in 900 ml of pH 6.8, 0.025 M sodium phosphate buffer at 37° C.

42. The stable solid dosage form of claim 41, wherein from about 49% to about 81% of upadacitinib is released from the solid dosage form in about 4 hours using USP I method at a rotation speed of 150 rpm in 900 ml pH 6.8, 0.025 M sodium phosphate buffer at 37° C.

43. The stable solid dosage form of any one of claims 34-36, wherein from about 58% to about 96% of upadacitinib is released from the solid dosage form in about 6 hours using USP I method at a rotation speed of 150 rpm in 900 ml of pH 6.8, 0.050 M sodium phosphate buffer at 37° C. and/or from about 65% to about 100% of upadacitinib is released from the solid dosage form in about 8 hours using USP I method at a rotation speed of 150 rpm in 900 ml of pH 6.8, 0.050 M sodium phosphate buffer at 37° C.

44. The stable solid dosage form of claim 43, wherein from about 47% to about 79% of upadacitinib is released from the solid dosage form in about 4 hours using USP I method at a rotation speed of 150 rpm in 900 ml pH 6.8, 0.050 M sodium phosphate buffer at 37° C.

45. The extended release solid dosage form of any one of claims 1-33 or the stable solid dosage form of any one of claims 34-44, wherein the upadacitinib or pharmaceutically acceptable salt thereof is present in the solid dosage form in an amount sufficient to deliver 5 mg to 50 mg, per unit dosage form, of upadacitinib free base equivalent.

46. The extended release solid dosage form of any one of claims 1-33 or the stable solid dosage form of any one of claims 34-44, wherein after storage the solid dosage form continues to retain (a) pharmaceutically acceptable levels of an upadacitinib degradation product and/or (b) a post-storage dissolution profile that is substantially similar to an initial dissolution profile.

47. The extended release solid dosage form or the stable solid dosage form of claim 46, wherein the solid dosage form comprises no more than 0.5% w/w of the upadacitinib degradation product during a shelf-life of the solid dosage form, wherein the shelf-life of the solid dosage form is about 6 months, about 12 months, about 18 months, about 24 months, about 30 months, or about 36 months.

48. The extended release solid dosage form or the stable solid dosage form of claim 46 or claim 47, wherein the upadacitinib degradation product is (3S,4R)-3-ethyl-4-(3-(hydroxymethyl)-3H-imidazo[1,2-a]pyrrolo[2,3-e]pyrazin-8-yl)-N-(2,2,2-trifluoroethyl) pyrrolidine-1-carboxamide.

49. The extended release solid dosage form or the stable solid dosage form of any one of claims 46-48, wherein storage of the solid dosage form is at 25° C.±2° C. and 60%±5% relative humidity for between about 3 months and about 48 months, about 6 months and about 36 months, or about 12 months and about 24 months.

50. The extended release solid dosage form or the stable solid dosage form of any one of claims 46-49, wherein the initial dissolution profile is not more than about 85% of upadacitinib released from the solid dosage form in about 1 or about 2 hours, from about 10% to about 65% of upadacitinib released from the solid dosage form in about 2 hours, from about 35% to about 90% of upadacitinib released from the solid dosage form in about 4 hours, and/or from about 70% to 100% of upadacitinib released from the solid dosage form in about 10 hours; and
wherein the initial dissolution profile is measured at 37° C.±2° C. using USP I apparatus at a rotation speed of 150 rpm in 900 mL of (1) pH 6.8, 0.025 M sodium phosphate buffer containing 2.75% sodium chloride, (2) pH 6.8, 0.025 M phosphate buffer, (3) pH 6.8, 0.050 M phosphate buffer, or (4) pH 1.1, 0.1N HCl.

51. The extended release solid dosage form or the stable solid dosage form of any one of claims 46-50, wherein the at least one pH dependent polymer is an enteric polymer or an anionic polysaccharide.

52. The extended release solid dosage form or the stable solid dosage form of any one of claims 46-51, wherein the at least one pH dependent polymer is hydroxypropylmethyl cellulose acetate succinate (HPMCAS) or alginic acid.

53. The extended release solid dosage form of any one of claims 1-33 or the stable solid dosage form of any one of claims 34-44, wherein the solid dosage form has a total weight less than 400 mg.

54. The extended release solid dosage form of claim 53, wherein the solid dosage form has a total weight between about 100 mg and about 300 mg.
